(19) United States
(12) Patent Application Publication
(10) Pub. No.: US 2024/0270743 A1
(43) Pub. Date: Aug. 15, 2024

(54) TETRAHYDROPYRAZOLOPYRIDINE-ANALOG LIGANDS OF NLRX1 AND USES THEREOF

(71) Applicant: LANDOS BIOPHARMA, INC., Roanoke, VA (US)

(72) Inventors: Josep Bassaganya-Riera, Blacksburg, VA (US); Raquel Hontecillas, Blacksburg, VA (US); Nuria Tubau-Juni, Blacksburg, VA (US); Andrew Leber, Blacksburg, VA (US)

(73) Assignee: LANDOS BIOPHARMA, INC., Roanoke, VA (US)

(21) Appl. No.: 18/682,563

(22) PCT Filed: Aug. 9, 2022
(86) PCT No.: PCT/US2022/039781

§ 371 Date: Feb. 9, 2024

Related U.S. Application Data
(60) Provisional application No. 63/231,992, filed on Aug. 11, 2021.

(51) Int. Cl. C07D 471/04; A61K 31/437; A61K 31/444; A61P 3/10; A61P 11/00; A61P 11/06

(52) U.S. Cl. CPC C07D 471/04; A61K 31/437; A61K 31/444; A61P 3/10; A61P 11/00; A61P 11/06

(57) ABSTRACT

Tetrahydropyrazolopyridine-analog compounds of Formula I:

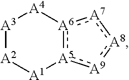

or pharmaceutically acceptable salts or esters thereof, which target the nucleotide-binding oligomerization domain, leucine rich repeat containing X1 (NLRX1) protein are described. Also described are methods of using the compounds in the treatment of chronic and/or inflammatory respiratory diseases, chronic and/or inflammatory diseases of the central nervous system, allergic diseases, autoimmune diseases, cardiovascular diseases, diabetes, hypereosinophilic syndrome, granulomatous disorders, cancer, and/or infectious diseases, among others. Exemplary conditions include asthma, chronic obstructive pulmonary disease, pulmonary fibrosis, Alzheimer's disease, atopic dermatitis, eosinophilic gastroenteritis, eosinophilic esophagitis, diabetes, and granulomatous disorders such as Churg-Strauss syndrome, berylliosis, and sarcoidosis.

### FIELD OF THE INVENTION

The present invention relates to tetrahydropyrazolopyridine-analog ligands of nucleotide-binding oligomerization domain, leucine rich repeat containing X1 (NLRX1), and uses thereof as medical treatments for diseases and disorders, particularly for treating and preventing inflammatory, allergic, or immune-mediated diseases.

### BACKGROUND

Nucleotide-binding oligomerization domain, leucine rich repeat containing X1 (NLRX1) (also called “NOD-like receptor X1” or “NLR family member X1” or “NOD9”) is a signaling pathway protein that is expressed in immune cells, the gastrointestinal tract, and skin, lung, muscle, endocrine, and reproductive tissues (Davis et al. 2014). The NLRX1 molecule has three distinct domains and localizes to the mitochondria (Arnoult et al. 2009). Published results indicate that the loss of NLRX1 worsens disease severity and alters immune cell metabolism (Leber et al. 2017) in models of inflammatory bowel disease (Leber et al. 2018, Lu et al. 2015, Soares et al. 2014). The NLRX1 protein has also been implicated in models of viral responses (Allen et al. 2011, Feng et al. 2017, Guo et al. 2016, Jaworska et al. 2014, Kim et al. 2017, Ma et al. 2017, Moore et al. 2008, Qin et al. 2017), bacterial infection (Philipson et al. 2015), fungal infection (Kale et al. 2017), cancer (Coutermarsh-Ott et al. 2016, Koblansky et al. 2016, Lei et al. 2016, Singh et al. 2015, Tattoli et al. 2016), hepatic steatosis (Kors et al. 2018, Wang et al. 2013), type 2 diabetes (Costford et al. 2018), brain injury (Theus et al. 2017), myocardial ischemia (Li et al. 2016), chronic obstructive pulmonary disease (Kang et al. 2015), and autoimmune encephalomyelitis (Eitas et al. 2014).

There are clear unmet clinical needs for safe, efficacious treatments for diseases in which NLRX1 is implicated. These include autoimmune diseases, inflammatory, and allergic diseases, such as asthma, chronic obstructive pulmonary disease, and infectious diseases. Due to low efficacy and poor safety, current allergy and autoimmune treatments require frequent monitoring, shifting treatment paradigms, and complex delivery methods. Thus, new treatments capable of being dosed orally for long-term management of disease are needed. In infectious diseases, high mutation rates in various microbes necessitate the development of novel non-antimicrobial treatments that spare the use of antibacterials, antifungals, and antivirals. Further, new strains and epidemic infections create a lag period between the emergence of a pathogen and the availability of microbe-specific interventions, creating a need for novel host-targeted therapeutics. Given the epidemic of infectious, allergic and autoimmune diseases as a whole, the NLRX1 pathway has the potential to significantly impact millions of patients.

Viral nucleic acids (Hong et al. 2012) and dietary lipids have been identified as natural ligands of NLRX1 (Lu et al. 2015). There is a need to develop novel ligands of the NLRX1 pathway to allow treatments to be tailored specifically to individual diseases and to potentially maximize their efficacy.

The present invention provides compounds that have been developed by novel medicinal chemistry approaches, and screened using in silico, in vitro, and in vivo techniques, to maximize their ability to bind to the NLRX1 protein and thus to induce a beneficial response in various disease conditions, including but not limited to, cancers, infectious diseases of bacterial, fungal and viral origin, and inflammatory, allergic, immune-mediated, or chronic respiratory diseases such as asthma, chronic obstructive pulmonary disease, and idiopathic pulmonary fibrosis, among others.

### SUMMARY OF THE INVENTION

The invention provides compounds of Formula I:

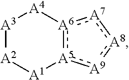

or pharmaceutically acceptable salts or esters thereof, wherein:
A¹ and A⁴ are each independently C(R¹)₂, N(R¹), O, S, N(R^{O}), C(R¹)(R^{O}), or C(═O);
A² is C(R¹)₂, N(R¹), O, or S;
A³ is N(Y), C(R¹)(Y), N(L^{Q}-Y), or C(R¹)(L^{Q}-Y);
A⁵ and A⁶ are each independently C, C(R¹), or N;
A⁷, A⁸, and A⁹ are each independently C(R¹)₂, N(R¹), O, S, C(R¹), N, N(R^{A}), C(R¹)(R^{A}), C(R^{A}), N(Z), C(R¹)(Z), C(Z), N(L^{Z}-Z), C(R¹)(LZ Z), or C(L^{Z}-Z), with the proviso that exactly one of A⁷, A⁸, and A⁹ is N(Z), C(R¹)(Z), C(Z), N(L^{Z}-Z), C(R¹)(L^{Z}-Z), or C(L^{Z}-Z);
R^{O} in each instance is independently hydroxyl, optionally substituted alkyloxy, thiol, optionally substituted alkylthio, or optionally substituted amino;
R^{A} is optionally substituted alkyl or hydroxyl;
L^{Q} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹);
L^{Z} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹);
Y is Y¹ or Y²;
Y¹ is:

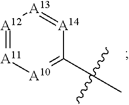

A¹⁰, A¹¹, A¹², A¹³, and A¹⁴ are each independently C(R¹), C(R^{Y}), or N, with the proviso that exactly one of A¹⁰, A¹¹, A¹², A¹³, and A¹⁴ is C(R^{Y});
Y² is:

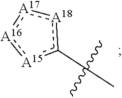

A¹⁵, A¹⁶, A¹⁷, and A¹⁸ are each independently C(R¹), C(R¹)₂, C(R^{Y}), C(R¹)(R^{Y}), N, N(R¹), N(R^{Y}), S, or O, with the proviso that exactly one of A¹⁵, A¹⁶, A¹⁷, and A¹⁸ is C(R^{Y}), C(R¹)(R^{Y}), or N(R^{Y});
R^{Y} is R^{L} or L^{Y}-R^{L};
R^{L} is hydroxyl, carboxyl, optionally substituted alkyloxy, thiol, sulfino, optionally substituted alkylthio, optionally substituted amino, optionally substituted alkyloxycarbonyl, optionally substituted carbamoyl, or optionally substituted sulfamoyl;
L^{Y} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹);
Z is:

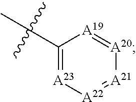

A¹⁹ and A²³ are each independently C(R¹) or N; A²⁰, A²¹, and A²² are each independently C(R¹), N, or C(R^{Z});
R^{Z} in each instance is independently halogen, optionally substituted alkyl, hydroxyl, optionally substituted alkyloxy, thiol, or optionally substituted alkylthio;
each - - - between adjacent atoms represents a bond that is present or absent;
R¹ in each instance is independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclic group.

In some embodiments, at least one of A¹ and A⁴ is C(R¹)(R^{O}), or C(═O). In some embodiments, A¹ is C(R¹)(R^{O}) or C(═O). In some embodiments, A¹ is C(═O). In some embodiments, A¹ is C(R¹)(R^{O}). In some embodiments, the R^{O} of A¹ is hydroxyl. In some embodiments, A¹ is C(R¹)₂. In some embodiments, A⁴ is C(R¹)(R^{O}), or C(═O). In some embodiments, A⁴ is C(═O). In some embodiments, A⁴ is C(R¹)(R^{O}). In some embodiments, the R^{O} of A⁴ is hydroxyl. In some embodiments, A⁴ is C(R¹)₂. In some embodiments, A² is C(R¹)₂. In some embodiments, A³ is N(Y) or N(L^{Q}-Y). In some embodiments, L^{Q} is not an oxygen atom, a sulfur atom, or N(R¹) when A³ is N(L^{Q}-Y). In some embodiments, A³ is N(Y) or C(R¹)(Y). In some embodiments, A³ is N(Y). In some embodiments, A⁵ is C. In some embodiments, A⁶ is C. In some embodiments, A⁵ and A⁶ are each C. In some embodiments, A⁷ is C(R¹) or C(R^{A}). In some embodiments, A⁷ is C(R¹). In some embodiments, A⁷ is C(R^{A}). In some embodiments, RA in each instance is unsubstituted C1-C4 alkyl, halogen-substituted C1-C4 alkyl, or hydroxyl. In some embodiments, R^{A} in each instance is unsubstituted C1-C4 alkyl. In some embodiments, A⁸ and A⁹ are each independently N(R¹), N, N(Z), or N(L^{Z}-Z). In some embodiments, A⁸ and A⁹ are each independently N, N(Z), or N(L^{Z}-Z), with the proviso that exactly one of A⁸ and A⁹ is N(Z) or N(L^{Z}-Z). In some embodiments, A⁸ is N(Z) or N(L^{Z}-Z). In some embodiments, A⁹ is N(Z) or N(L^{Z}-Z). In some embodiments, L^{Z} is not an oxygen atom, a sulfur atom, or N(R¹) when the exactly one of A⁷, A⁸, or A⁹ is N(L^{Z}- Z). In some embodiments, A⁸ and A⁹ are each independently N or N(Z), with the proviso that exactly one of A⁸ and A⁹ is N(Z). In some embodiments, A⁸ is N(Z). In some embodiments, A⁹ is N(Z).

In some embodiments, Y is Y¹. In some embodiments, at least one of A¹⁰, A¹¹, A¹², A¹³, and A¹⁴ is N. In some embodiments, exactly one of A¹⁰, A¹¹, A¹², A¹³, and A¹⁴ is N. In some embodiments, A¹⁰ is N. In some embodiments, A¹¹ is N. In some embodiments, A¹² is N. In some embodiments, A¹³ is N. In some embodiments, A¹⁴ is N. In some embodiments, A¹⁰ and A¹³ are N. In some embodiments, A¹² and A¹⁴ are N. In some embodiments, exactly one of A¹¹ and A¹² is C(R^{Y}). In some embodiments, A¹¹ is C(R^{Y}). In some embodiments, A¹² is C(R^{Y}). In some embodiments, A¹³ is N. In some embodiments, each of A¹⁰, A¹¹, A¹², A¹³, and A¹⁴ is C(R¹) unless specified otherwise.

In some embodiments, Y is Y². In some embodiments, exactly one of A¹⁵, A¹⁶, A¹⁷, and A¹⁸ is N(R¹), N(R^{Y}), S, or O. In some embodiments, at least one of A¹⁵, A¹⁶, A¹⁷, and A¹⁸ is N, N(R¹), N(R^{Y}), S, or O. In some embodiments, at least two of A¹⁵, A¹⁶, A¹⁷, and A¹⁸ are each independently N, N(R¹), N(R^{Y}), S, or O. In some embodiments, exactly one of A¹⁵, A¹⁶, and A¹⁷ is C(R^{Y}) or N(R^{Y}). In some embodiments, A¹⁵ is N. In some embodiments, A¹⁵ is C(R¹). In some embodiments, A¹⁶ is C(R^{Y}). In some embodiments A¹⁶ is N(R^{Y}). In some embodiments, A¹⁷ is N(R¹), S, or O. In some embodiments, A¹⁷ is N(R¹). In some embodiments, A¹⁷ is O. In some embodiments, A¹⁷ is N. In some embodiments, A¹⁸ is N. In some embodiments, A¹⁶ is N(R^{Y}), A¹⁷ is N, and A¹⁸ is N.

In some embodiments, R^{Y} is R^{L}. In some embodiments, R^{Y} is L^{Y}-R^{L}. In some embodiments, R¹. is hydroxyl, carboxyl, optionally substituted alkyloxy, optionally substituted amino, optionally substituted alkyloxycarbonyl, and optionally substituted carbamoyl. In some embodiments, RI is hydroxyl, carboxyl, unsubstituted C1-C4 alkyloxy, unsubstituted amino, amino substituted with one or two C1-C4 alkyl groups, unsubstituted C1-C4 alkyloxycarbonyl, unsubstituted carbamoyl, and carbamoyl comprising an amino substituted with one or two C1-C4 alkyl groups. In some embodiments, R^{L} is carboxyl. In some embodiments, L^{Y} is not an oxygen atom, a sulfur atom, or N(R¹) when the exactly one of A¹⁵, A¹⁶, A¹⁷, and A¹⁸ is N(R^{Y}). In some embodiments, L^{Y} is optionally substituted alkylene optionally containing one or two heteroatom(s). In some embodiments, L^{Y} is optionally substituted alkylene optionally containing one or two heteroatom(s) with a contiguous backbone chain of no more than four atoms. In some embodiments, L^{Y} is optionally substituted alkylene with a contiguous backbone chain of no more than four atoms. In some embodiments, L^{Y} is unsubstituted C1, C2, C3, or C4 alkylene.

In some embodiments, at least one of A¹⁹, A²⁰, A²¹, A²², and A²³ is N or at least one of A²⁰, A²¹, and A²² is C(R^{Z}). In some embodiments, at least one of A¹⁹, A²⁰, A²¹, A²², and A²³ is N. In some embodiments, A¹⁹ is N. In some embodiments, A²⁰ is N. In some embodiments, A²¹ is N. In some embodiments, A²⁰ and A²² are each N. In some embodiments, at least one of A²⁰, A²¹, and A²² is C(R^{Z}). In some embodiments, at least two of A²⁰, A²¹, and A²² are C(R^{Z}). In some embodiments, A²⁰ is C(R^{Z}). In some embodiments, A²¹ is C(R²). In some embodiments, A²² is C(R²). In some embodiments, A¹⁹ and A²³ are each C(R¹).

In some embodiments, R² in each instance is independently halogen, optionally substituted alkyl, hydroxyl, or optionally substituted alkyloxy. In some embodiments, R² in each instance is independently halogen, unsubstituted C1-C4 alkyl, halogen-substituted C1-C4 alkyl, hydroxyl, or unsubstituted C1-C4 alkyloxy. In some embodiments, R² in each instance is halogen.

In some embodiments, R¹ in each instance, except where specified otherwise, is independently hydrogen, halogen, optionally substituted alkyl, optionally substituted cycloalkyl, optionally substituted alkyloxy, optionally substituted cycloalkyloxy, optionally substituted alkylthio, optionally substituted alkylsulfinyl, optionally substituted cycloalkylthio, optionally substituted cycloalkylsulfinyl, optionally substituted amino, acyl, optionally substituted aryl, optionally substituted aryloxy, optionally substituted arylthio, optionally substituted heteroaryl, optionally substituted heteroaryloxy, optionally substituted heteroarylthio, optionally substituted heteroarylsulfinyl, or an optionally substituted non-aromatic heterocyclic group. In some embodiments, R¹ in each instance, except where specified otherwise, is independently hydrogen, halogen, unsubstituted alkyl, unsubstituted cycloalkyl, unsubstituted alkyloxy, unsubstituted cycloalkyloxy, unsubstituted alkylthio, unsubstituted alkylsulfinyl, unsubstituted cycloalkylthio, unsubstituted cycloalkylsulfinyl, unsubstituted amino, acyl, unsubstituted aryl, unsubstituted aryloxy, unsubstituted arylthio, unsubstituted heteroaryl, unsubstituted heteroaryloxy, unsubstituted heteroarylthio, unsubstituted heteroarylsulfinyl, or an unsubstituted non-aromatic heterocyclic group. In some embodiments, R¹ in each instance, except where specified otherwise, is independently hydrogen, halogen, or unsubstituted C1-C6 alkyl. In some embodiments, R¹ in each instance, except where specified otherwise, is independently hydrogen or halogen. In some embodiments, R¹ in each instance, except where specified otherwise, is hydrogen.

In some embodiments, the compound has the structure of any compound shown in FIGS. 1A-1R, or a pharmaceutically acceptable salt or ester thereof. In some embodiments, the compound has the structure of any compound shown in FIGS. 2A-2E, or a pharmaceutically acceptable salt or ester thereof. In some embodiments, the compound has the structure of:

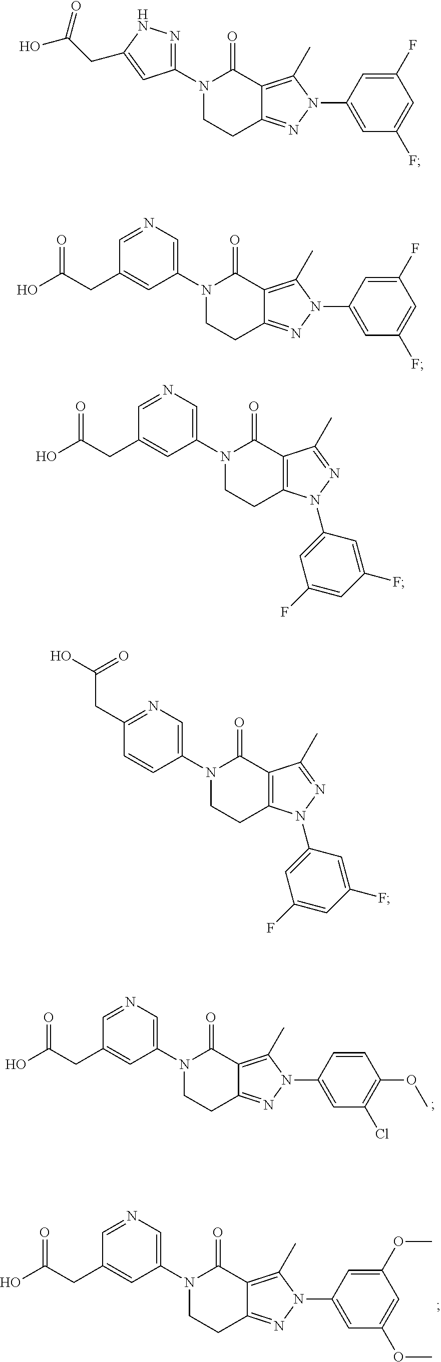 [SMILES: Cc1c2c(nn1-c1cc(F)cc(F)c1)CCN(c1cc(CC(=O)O)[nH]n1)C2=O ; Cc1c2c(nn1-c1cc(F)cc(F)c1)CCN(c1cncc(CC(=O)O)c1)C2=O ; Cc1nn(-c2cc(F)cc(F)c2)c2c1C(=O)N(c1cncc(CC(=O)O)c1)CC2 ; Cc1nn(-c2cc(F)cc(F)c2)c2c1C(=O)N(c1ccc(CC(=O)O)nc1)CC2 ; COc1ccc(-n2nc3c(c2C)C(=O)N(c2cncc(CC(=O)O)c2)CC3)cc1Cl ; COc1cc(OC)cc(-n2nc3c(c2C)C(=O)N(c2cncc(CC(=O)O)c2)CC3)c1]

or a pharmaceutically acceptable salt or ester thereof.

The invention also provides methods of treating a condition in an animal with a compound as described herein. The methods comprise administering an effective amount of the compound to the animal. The condition can comprise at least one of a chronic and/or inflammatory respiratory disease, a chronic and/or inflammatory disease of the central nervous system, an allergic disease, an autoimmune disease, a cardiovascular disease, diabetes, hypereosinophilic syndrome, a granulomatous disorder, cancer, and an infectious disease, among others. Exemplary conditions include asthma, chronic obstructive pulmonary disease, pulmonary fibrosis, Alzheimer's disease, atopic dermatitis, eosinophilic gastroenteritis, eosinophilic esophagitis, diabetes, and granulomatous disorders such as Churg-Strauss syndrome, berylliosis, and sarcoidosis.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1R. Computational prediction of binding of selected compounds to NLRX1 in kcal/mol.

FIGS. 2A-2E. Exemplary compounds of the invention: LABP-72-4 (FIG. 2A); 1,2,3-triazole analog of LABP-72-4 (FIG. 2B); LABP-72-38 (FIG. 2C); LABP-72-56 (FIG. 2D); LABP-72-69 (FIG. 2E).

FIGS. 3A and 3B. Immunological validation of LABP-72-4, LABP-72-38, LABP-72-56, and LABP-72-69 activity in CD4+ T cells. Percentages of TNFα+ (FIG. 3A) and IFNγ+ (FIG. 3B) CD4+ T cells were measured by flow cytometry after in vitro treatment of cells with LABP compounds at concentrations of 100 nanomolar. Statistical significance (p<0.05) is marked by asterisks.

FIGS. 4A and 4 B. In vivo validation of LABP-72-38 efficacy in an OVA-induced model of asthma. Flow cytometry measure of eosinophils (FIG. 4A) within the lung and plasma concentrations of anti-OVA IgE (FIG. 4B) after 4 days of OVA challenge and treatment with vehicle or LABP-72-38 (50 mg/kg) daily by oral gavage. Statistical significance (p<0.05) is marked by asterisks.

FIGS. 5A and 5B. In vivo validation of LABP-72-38 efficacy in a house dust mite model of asthma. Flow cytometry measure of eosinophils (FIG. 5A) and neutrophils (FIG. 5B) within the lung after 3 weeks of house dust mite challenge and treatment with vehicle or LABP-72-38 (20 mg/kg) daily by intranasal route. Statistical significance (p<0.05) is marked by asterisks.

FIGS. 6A and 6B. In vivo validation of LABP-72-38 efficacy in a bleomycin mouse model of pulmonary fibrosis. Flow cytometry measure of neutrophils (FIG. 6A) and IL21+ cells (FIG. 6B) within the lung 2 weeks after bleomycin challenge and treatment with vehicle or LABP-72-38 (50 mg/kg) daily for 1 week by oral gavage. Statistical significance (p<0.05) is marked by asterisks.

FIGS. 7A and 7B. In vivo validation of LABP-72-38 efficacy in a high fat diet induced obesity model. Blood glucose concentration (FIG. 7A) and body weight-normalized liver weight (FIG. 7B) after 16 weeks of high fat diet feeding in mice treated with vehicle or LABP-72-38 (10 mg/kg) daily by oral gavage for 4 weeks. Statistical significance (p<0.05) is marked by asterisks.

### DETAILED DESCRIPTION OF THE INVENTION

The term “halogen” refers to fluorine, chlorine, bromine, and iodine. Fluorine, chlorine, and bromine are preferred.

The term “hetero atom” refers to an oxygen atom, a sulfur atom, and a nitrogen atom.

The term “alkyl” includes a monovalent straight or branched hydrocarbon group having one to eight carbon atom(s). Examples include methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. C1-C6 alkyl is preferred. C1-C4 alkyl or C1-C3 alkyl is further preferred. When a number of carbons is specified, it means “alkyl” having the carbon number within the range.

The term “alkenyl” includes a monovalent straight or branched hydrocarbon group having two to eight carbon atoms and one or more double bond(s). Examples include vinyl, allyl, 1-propenyl, 2-butenyl, 2-pentenyl, 2-hexenyl, 2-heptenyl, 2-octenyl, and the like. C2-C6 alkenyl is preferred. C2-C4 alkenyl is further preferred.

The term “alkynyl” includes a monovalent straight or branched hydrocarbon group having two to eight carbon atoms and one or more triple bond(s). Examples include ethynyl, 1-propynyl, 2-propynyl, 2-butynyl, 2-pentynyl, 2-hexynyl, 2-heptynyl, 2-octynyl, and the like. C2-C6 alkynyl is preferred. C2-C4 alkynyl is further preferred.

The term “cycloalkyl” includes a cycloalkyl having three to eight carbon atoms. Examples include cyclopropyl, cyclobutyl, cyclopentyl, cyclohexyl, cycloheptyl, cyclooctyl, and the like. C3-C6 cycloalkyl is preferred.

The term “cycloalkenyl” includes a cycloalkenyl having three to eight carbon atoms. Examples include cyclopropenyl, cyclobutenyl, cyclopentenyl, cyclohexenyl, cycloheptenyl, cycloocentyl, and the like. C3-C6 cycloalkenyl is preferred.

The term “alkyloxy” includes a group wherein an oxygen atom is substituted with one “alkyl” as described herein. Examples include methyloxy, ethyloxy, n-propyloxy, isopropyloxy, n-butyloxy, isobutyloxy, sec-butyloxy, tert-butyloxy, n-pentyloxy, isopentyloxy, 2-pentyloxy, 3-pentyloxy, n-hexyloxy, isohexyloxy, 2-hexyloxy, 3-hexyloxy, n-heptyloxy, n-octyloxy, and the like. C1-C6 alkyloxy is preferred. C1-C4 alkyloxy or C1-C3 alkyloxy is further preferred. When a number of carbons is specified, it means “alkyloxy” having the carbon number within the range.

The term “alkenyloxy” includes a group wherein an oxygen atom is substituted with one “alkenyl” as described herein. Examples include vinyloxy, allyloxy, 1-propenyloxy, 2-butenyloxy, 2-pentenyloxy, 2-hexenyloxy, 2-heptenyloxy, 2-octenyloxy, and the like. C2-C6 alkenyloxy is preferred. Moreover, C2-C4 alkenyloxy is further preferred. When a number of carbons is specified, it means “alkenyloxy” having the carbon number within the range.

The term “alkynyloxy” includes a group wherein an oxygen atom is substituted with one “alkynyl” as described herein. Examples include cthynyloxy, 1-propynyloxy, 2-propynyloxy, 2-butynyloxy, 2-pentynyloxy, 2-hexynyloxy, 2-heptynyloxy, 2-octynyloxy, and the like. C2-C6 alkynyloxy is preferred. C2-C4 alkynyloxy is further preferred. When a number of carbons is specified, it means “alkynyloxy” having the carbon number within the range.

The term “cycloalkyloxy” includes a group wherein an oxygen atom is substituted with one “cycloalkyl” as described herein. Examples include cyclopropyloxy, cyclobutyloxy, cyclopentyloxy, cyclohexyloxy, cycloheptyloxy, and cyclooctyloxy. C3-C6 cycloalkyloxy is preferred. When a number of carbons is specified, it means “cycloalkyloxy” having the carbon number within the range.

The term “cycloalkenyloxy” includes a group wherein an oxygen atom is substituted with one “cycloalkenyl” as described herein. Examples include cyclopropenyloxy, cyclobutenyloxy, cyclopentenyloxy, cyclohexenyloxy, cycloheptenyloxy, and cyclooctenyloxy. C3-C6 cycloalkenyloxy is preferred. When a number of carbons is specified, it means “cycloalkenyloxy” having the carbon number within the range.

The term “alkylthio” includes a group wherein a sulfur atom is substituted with one “alkyl” as described herein. Examples include methylthio, ethylthio, n-propylthio, isopropylthio, n-butylthio, isobutylthio, sec-butylthio, tert-butylthio, n-pentylthio, isopentylthio, 2-pentylthio, 3-pentylthio, n-hexylthio, isohexylthio, 2-hexylthio, 3-hexylthio, n-heptylthio, n-octylthio, and the like. C1-C6 Alkylthio is preferred. C1-C4 alkylthio is further preferred. C1-C3 alkylthio is further preferred. When a number of carbons is specified, it means “alkylthio” having the carbon number within the range.

The term “alkenylthio” includes a group wherein a sulfur atom is substituted with one “alkenyl” as described herein. Examples include vinylthio, allylthio, 1-propenylthio, 2-butenylthio, 2-pentenylthio, 2-hexenylthio, 2-heptenylthio, 2-octenylthio, and the like. C2-C6 Alkenylthio is preferred. C2-C4 alkylthio is further preferred. When a number of carbons is specified, it means “alkenylthio” having the carbon number within the range.

The term “alkynylthio” includes a group wherein a sulfur atom is substituted with one “alkynyl” as described herein. Examples include ethynylthio, 1-propynylthio, 2-propynylthio, 2-butynylthio, 2-pentynylthio, 2-hexynylthio, 2-heptynylthio, 2-octynylthio, and the like. C2-C6 alkynylthio is preferred. C2-C4 alkynylthio is further preferred. When a number of carbons is specified, it means “alkynylthio” having the carbon number within the range.

The term “alkylsulfinyl” includes a group wherein sulfinyl is substituted with one “alkyl” as described herein. Examples include methylsulfinyl, ethylsulfinyl, n-propylsulfinyl, isopropylsulfinyl, n-butylsulfinyl, isobutylsulfinyl, sec-butylsulfinyl, tert-butylsulfinyl, n-pentylsulfinyl, isopentylsulfinyl, 2-pentylsulfinyl, 3-pentylsulfinyl, n-hexylsulfinyl, isohexylsulfinyl, 2-hexylsulfinyl, 3-hexylsulfinyl, n-heptylsulfinyl, n-octylsulfinyl, and the like. C1-C6 alkylsulfinyl is preferred. C1-C4 alkylsulfinyl is further preferred.

The term “alkylsulfonyl” includes a group wherein sulfonyl is substituted with one “alkyl” as described herein. Examples include methylsulfonyl, ethylsulfonyl, n-propylsulfonyl, isopropylsulfonyl, n-butylsulfonyl, isobutylsulfonyl, sec-butylsulfonyl, tert-butylsulfonyl, n-pentylsulfonyl, isopentylsulfonyl, 2-pentylsulfonyl, 3-pentylsulfonyl, n-hexylsulfonyl, isohexylsulfonyl, 2-hexylsulfonyl, 3-hexylsulfonyl, n-heptylsulfonyl, n-octylsulfonyl, and the like. C1-C6 alkylsulfonyl is preferred. C1-C4 alkylsulfonyl is further preferred.

The term “alkylsulfonyloxy” includes a group wherein an oxygen atom is substituted with one “alkylsulfonyl” as described herein. Examples include methylsulfonyloxy, ethylsulfonyloxy, n-propylsulfonyloxy, isopropylsulfonyloxy, n-butylsulfonyloxy, isobutylsulfonyloxy, sec-butylsulfonyloxy, tert-butylsulfonyloxy, n-pentylsulfonyloxy, isopentylsulfonyloxy, 2-pentylsulfonyloxy, 3-pentylsulfonyloxy, n-hexylsulfonyloxy, isohexylsulfonyloxy, 2-hexylsulfonyloxy, 3-hexylsulfonyloxy, n-heptylsulfonyloxy, n-octylsulfonyloxy, and the like. C1-C6 alkylsulfonyl is preferred. C1-C4 alkylsulfonyl is further preferred.

The term “cycloalkylthio” includes a group wherein a sulfur atom is substituted with one “cycloalkyl” as described herein. Examples include cyclopropylthio, cyclobutylthio, cyclopentylthio, cyclohexylthio, cycloheptylthio, cyclooctylthio, and the like. C3-C6 cycloalkylthio is preferred. When a number of carbons is specified, it means “cycloalkylthio” having the carbon number within the range.

The term “cycloalkylsulfinyl” includes a group in which sulfinyl is substituted with one “cycloalkyl” as described herein. Examples include cyclopropylsulfinyl, cyclobutylsulfinyl, cyclopentylsulfinyl, cyclohexylsulfinyl, cycloheptylsulfinyl, and cyclooctylsulfinyl are exemplified. Preferably C3-C6 cycloalkylsulfinyl.

The term “cycloalkylsulfonyl” includes a group in which sulfonyl is substituted with one “cycloalkyl” as described herein. Examples include cyclopropylsulfonyl, cyclobutylsulfonyl, cyclopentylsulfonyl, cyclohexylsulfonyl, cycloheptylsulfonyl, and cyclooctylsulfonyl. C3-C6 cycloalkylsulfonyl is preferred.

The term “cycloalkylsulfonyloxy” includes a group in which an oxygen atom is substituted with one “cycloalkylsulfonyl” as described herein. Examples include cyclopropylsulfonyloxy, cyclobutylsulfonyloxy, cyclopentyl sulfonyloxy, cyclohexyl sulfonyloxy, cycloheptylsulfonyloxy, and cyclooctylsulfonyloxy. C6-C3 cycloalkylsulfonyloxy is preferred.

The term “cycloalkenylthio” includes a group in which a sulfur atom is substituted with one “cycloalkenyl” as described herein. Examples include cyclopropenylthio, cyclobutenylthio, cyclopentenylthio, cyclohexenylthio, cycloheptenylthio, and cyclooctenylthio. C3-C6 cycloalkenylthio is preferred. When a number of carbons is specified, it means “cycloalkenylthio” having the carbon number within the range.

The term “cycloalkenylsulfinyl” includes a group in which sulfinyl is substituted with one “cycloalkenyl” as described herein. Examples include cyclopropenylsulfinyl, cyclobutenylsulfinyl, cyclopentenylsulfinyl, cyclohexenylsulfinyl, cycloheptenylsulfinyl, and cyclooctenylsulfinyl. C3-C6 cycloalkenylsulfinyl is preferred.

The term “cycloalkenylsulfonyl” includes a group in which sulfonyl is substituted with one “cycloalkenyl” as described herein. Examples include cyclopropenylsulfonyl, cyclobutenylsulfonyl, cyclopentenylsulfonyl, cyclohexenylsulfonyl, cycloheptenylsulfonyl, and cyclooctenylsulfonyl. Preferably C3-C6 cycloalkenylsulfonyl is preferred.

The term “cycloalkenylsulfonyloxy” includes a group in which an oxygen atom is substituted with one “cycloalkenylsulfonyl” described as described herein. Examples include cyclopropenylsulfonyloxy, cyclobutenylsulfonyloxy, cyclopentenylsulfonyloxy, cyclohexenylsulfonyloxy, cycloheptenylsulfonyloxy, and cyclooctenylsulfonyloxy. C3-C6 cycloalkenylsulfonyloxy is preferred.

The term “alkyloxycarbonyl” includes a group in which carbonyl is substituted with one “alkyloxy” as described herein. Examples include methyloxycarbonyl, ethyloxycarbonyl, n-propyloxycarbonyl, isopropyloxycarbonyl, n-butyloxycarbonyl, tert-butyloxycarbonyl, and n-pentyloxycarbonyl. C1-C6, C1-C4, or C1-C3 alkyloxycarbonyl is preferred. C1-C2 alkyloxycarbonyl is further preferred.

The term “alkenyloxycarbonyl” includes a group in which carbonyl is substituted with one “alkenyloxy” as described herein. Examples include vinyloxycarbonyl, allyloxycarbonyl, 1 -propenyloxycarbonyl, 2-butenyloxycarbonyl, and 2-pentenyloxyarbonyl. C2-C6 or C2-C4 alkyloxycarbonyl is preferred.

The term “alkynyloxycarbonyl” includes a group in which carbonyl is substituted with one “alkynyloxy” as described herein. Examples include ethynyloxycarbonyl, 1-propynyloxycarbonyl, 2-propynyloxycarbonyl, 2-butynyloxyarbonyl, and 2-pentynyloxycarbonyl. C2-C6 or C2-C4 alkynyloxycarbonyl is preferred.

The term “acyl” includes alkylcarbonyl wherein the part of alkyl is “alkyl” as described hercin, alkenylcarbonyl wherein the part of alkenyl is “alkenyl” as described herein, alkynylcarbonyl wherein the part of alkynyl is “alkynyl” as described herein, cycloalkylcarbonyl wherein the part of cycloalkyl is “cycloalkyl” as described herein, arylcarbonyl wherein the part of aryl is “aryl” as described herein, heteroarylcarbonyl wherein the part of heteroaryl is “heteroaryl” as described herein, and non-aromatic heterocycliccarbonyl wherein the part of non-aromatic heterocyclic group is “non-aromatic heterocyclic group” as described herein. “Alkyl,” “alkenyl,” “alkynyl,” “cycloalkyl,” “aryl,” “heteroaryl,” and “non-aromatic heterocyclic group” may be substituted respectively with substituent groups exemplified in “optionally substituted alkyl,” “optionally substituted alkenyl,” “optionally substituted alkynyl,” “optionally substituted cycloalkyl,” “optionally substituted aryl,” “optionally substituted heteroaryl,” and “optionally substituted non-aromatic heterocyclic group” as described herein. Examples of the acyl group include acetyl, propionyl, butyroyl, cyclohexylcarbonyl, benzoyl, pyridinecarbonyl, and the like.

The term “optionally substituted amino” includes an amino group which may be substituted with one or two group(s) of “alkyl” as described herein, “alkenyl” as described herein, “alkynyl” as described herein, “cycloalkyl” as described herein, “cycloalkynyl” as described herein, “aryl” as described herein, “heteroaryl” as described herein, “acyl” as described herein, “alkyloxycarbonyl” as described herein, “alkenyloxycarbonyl” as described herein, “alkynyloxycarbonyl” as described herein, “alkyl sulfonyl,” “alkenylsulfonyl,” “alkynylsulfonyl,” “arylsulfonyl,” and/or “heteroarylsulfonyl” as described herein. Examples of the optionally substituted amino group include amino, methylamino, dimethylamino, ethylamino, diethylamino, ethylmethylamino, benzylamino, acetylamino, benzoylamino, methyloxycarbonylamino, and methanesulfonylamino. Amino, methylamino, dimethylamino, ethylmethylamino, diethylamino, acetylamino, and methanesulfonylamino are preferred.

The term “optionally substituted carbamoyl” includes an aminocarbonyl group wherein the part of optionally substituted amino is “optionally substituted amino” as described herein. Examples of the optionally substituted carbamoyl group includes carbamoyl, N-methylcarbamoyl, N,N-dimethylcarbamoyl, N-ethyl-N-methylcarbamoyl, N,N-dicthylcarbamoyl, N-phenylcarbamoyl, N-benzylcarbamoyl, N-acetylcarbamoyl, and N-methylsulfonylcarbamoyl etc. Carbamoyl, N-methylcarbamoyl, N,N-dimethylcarbamoyl, and N-methylsulfonylcarbamoyl etc. are preferred.

The term “optionally substituted sulfamoyl” includes an aminosulfonyl group wherein the part of optionally substituted amino is “optionally substituted amino” as described herein. Examples of the optionally substituted sulfamoyl group include sulfamoyl, N-methylsulfamoyl, N,N-dimethylsulfamoyl, N-ethyl-N-methyl sulfamoyl, N,N-dicthylsulfamoyl, N-phenylsulfamoyl, N-benzylsulfamoyl, N-acetylsulfamoyl, and N-methylsulfonylsulfamoyl etc. Sulfamoyl, N-methylsulfamoyl, N,N-dimethylsulfamoyl, and N-methylsulfonylsulfamoyl etc. are preferred.

The term “alkylene” means a straight or branched alkylene group having one to eight carbon atom(s). Examples include methylene, ethylene, 1-methylethylene, trimethylene, 1-methyltrimethylene, pentamethylene, hexamethylene, and the like. C1-C4 or C1-3 alkylenes are preferred. C1-C2 alkylene is further preferred.

The term “aryl” includes an aromatic monocyclic or aromatic fused cyclic hydrocarbons. It may be fused with “cycloalkyl” as described herein, “cycloalkenyl” as described herein or “non-aromatic heterocyclic group” as described herein at any possible position. Both of monocyclic ring and fused ring may be substituted at any position. Examples include phenyl, 1 -naphthyl, 2-naphthyl, anthryl, tetrahydronaphthyl, 1,3-benzodioxolyl, 1,4-benzodioxanyl etc. Phenyl, 1-naphthyl, and 2-naphthyl are preferred. Phenyl is further preferred.

The term “non-aromatic heterocyclic group” includes a 5- to 7-membered non-aromatic heterocyclic ring containing one or more of heteroatom(s) selected independently from oxygen, sulfur, and nitrogen atoms or a multicyclic ring formed by fusing the two or more rings thereof. Examples include pyrrolidinyl (e.g., 1-pyrrolidinyl, 2-pyrrolidinyl), pyrrolinyl (e.g., 3-pyrrolinyl), imidazolidinyl (e.g., 2-imidazolidinyl), imidazolinyl (e.g., imidazolinyl), pyrazolidinyl (e.g., 1-pyrazolidinyl, 2-pyrazolidinyl), pyrazolinyl (e.g., pyrazolinyl), piperidyl (e.g., piperidino, 2-piperidyl), piperazinyl (e.g., 1-piperazinyl), indolinyl (e.g., 1-indolinyl), isoindolinyl (e.g., isoindolinyl), morpholinyl (e.g., morpholino, 3-morpholinyl) etc.

The term “heteroaryl” includes a 5- to 6-membered aromatic ring containing one or more of heteroatom(s) selected independently from oxygen, sulfur, and nitrogen atoms. It may be fused with “cycloalkyl” as described herein, “aryl” as described herein, “non-aromatic heterocyclic group” as described herein, or other heteroaryl at any possible position. The heteroaryl group may be substituted at any position whenever it is a monocyclic ring or a fused ring. Examples include pyrrolyl (e.g., 1-pyrrolyl, 2-pyrrolyl, 3-pyrrolyl), furyl (e.g., 2-furyl, 3-furyl), thienyl (e.g., 2-thienyl, 3-thicnyl), imidazolyl (e.g., 2-imidazolyl, 4-imidazolyl), pyrazolyl (e.g., 1-pyrazolyl, 3-pyrazolyl), isothiazolyl (e.g., 3-isothiazolyl), isoxazolyl (e.g., 3-isoxazolyl), oxazolyl (e.g., 2-oxazolyl), thiazolyl (e.g., 2-thiazolyl), pyridyl (e.g., 2-pyridyl, 3-pyridyl, 4-pyridyl), pyrazinyl (e.g., 2-pyrazinyl), pyrimidinyl (e.g., 2-pyrimidinyl, 4-pyrimidinyl), pyridazinyl (e.g., 3-pyridazinyl), tetrazolyl (e.g., 1H-tetrazolyl), oxadiazolyl (e.g., 1,3,4-oxadiazolyl), thiadiazolyl (e.g., 1,3,4-thiadiazolyl), indolidinyl (e.g., 2-indolidinyl, 6-indolidinyl), isoindolynyl (e.g., 2-isoindolynyl), indolyl (e.g., 1-indolyl, 2-indolyl, 3-indolyl), indazolyl (e.g., 3-indazolyl), purinyl (e.g., 8-purinyl), quinolidinyl (e.g., 2-quinolidinyl), isoquinolyl (e.g., 3-isoquinolyl), quinolyl (e.g., 2-quinolyl, 5-quinolyl), phtharazinyl (e.g., 1-phtharazinyl), naphthylidinyl (e.g., 2-naphthylidinyl), quinolanyl (e.g., 2-quinolanyl), quinazolinyl (e.g., 2-quinazolinyl), cinnolinyl (e.g., 3-cinnolinyl), pteridinyl (e.g., 2-pteridinyl), carbazolyl (e.g., 2-carbazolyl, 4-carbazolyl), phenanthridinyl (e.g., 2-phenanthridinyl, 3-phenanthridinyl), acridinyl (e.g., 1-acridinyl, 2-acridinyl), dibenzofuranyl (e.g., 1-dibenzofuranyl, 2-dibenzofuranyl), benzoimidazolyl (e.g., 2-benzoimidazolyl), benzoisoxazolyl (e.g., 3-benzoisoxazolyl), benzooxazolyl (e.g., 2-benzooxazolyl), benzooxadiazolyl (e.g., 4-benzooxadiazolyl), benzoisothiazolyl (e.g., 3-benzoisothiazolyl), benzothiazolyl (e.g., 2-benzothiazolyl), benzofuryl (e.g., 3-benzofuryl), benzothienyl (e.g., 2-benzothicnyl), dibenzothienyl (e.g., 2-dibenzothienyl), and benzodioxolyl (e.g., 1,3-benzodioxolyl), etc.

The term “aryloxy” includes a group in which an oxygen atom is substituted with one “aryl” as described herein. Examples include phenyloxy and naphthyloxy, etc.

The term “arylthio” includes a group in which a sulfur atom is substituted with one “aryl” as described herein. Examples include phenylthio and naphthylthio, etc.

The term “arylsulfinyl” includes a group in which sulfinyl is substituted with one “aryl” as described herein. Examples include phenylsulfinyl and naphthylsulfinyl, etc.

The term “arylsulfonyl” includes a group in which sulfonyl is substituted with one “aryl” as described herein. Examples include phenylsulfonyl and naphthylsulfoinyl, etc.

Examples of “arylsulfonyloxy” include phenylsulfonyloxy and naphthylsulfonyloxy, etc.

The term “aryloxycarbonyl” includes a group in which carbonyl is substituted with one “aryloxy” as described herein. Examples include phenyloxycarbonyl, 1-naphthyloxycarbonyl and 2-naphthyloxycarbonyl, etc.

The term “heteroaryloxy” includes a group in which an oxygen atom is substituted with one “heteroaryl” as described herein. Examples include pyrrolyloxy, furyloxy, thienyloxy, imidazolyloxy, pyrazolyloxy, isothiazolyloxy, isoxazolyloxy, oxazolyloxy, thiazolyloxy, pyridyloxy, pyrazinyloxy, pyrimidinyloxy, pyridazinyloxy, tetrazolyloxy, oxadiazolyloxy, thiadiazolyloxy, indolidinyloxy, isoindolynyloxy, indolyloxy, indazolyloxy, purinyloxy, quinolidinyloxy, isoquinolyloxy, quinolyloxy, phtharazinyloxy, naphthylidinyloxy, quinolanyloxy, quinazolinyloxy, cinnolinyloxy, pteridinyloxy, carbazolyloxy, phenanthridinyloxy, acridinyloxy, dibenzofuranyloxy, benzoimidazolyloxy, benzoisoxazolyloxy, benzooxazolyloxy, benzooxadiazolyloxy, benzoisothiazolyloxy, benzothiazolyloxy, benzofuryloxy, benzothienyloxy, dibenzothienyloxy, and benzodioxolyloxy are exemplified. Preferably furyloxy, thienyloxy, imidazolyloxy, pyrazolyloxy, isothiazolyloxy, isoxazolyloxy, oxazolyloxy, thiazolyloxy, pyridyloxy, pyrazinyloxy, pyrimidinyloxy, and pyridazinyloxy, etc.

The term “heteroarylthio” includes a group in which a sulfur atom is substituted with one “heteroaryl” as described herein. Examples include pyrrolylthio, furylthio, thienylthio, imidazolylthio, pyrazolylthio, isothiazolylthio, isoxazolylthio, oxazolylthio, thiazolylthio, pyridylthio, pyrazinylthio, pyrimidinylthio, pyridazinylthio, tetrazolylthio, oxadiazolylthio, thiadiazolylthio, indolidinylthio, isoindolynylthio, indolylthio, indazolylthio, purinylthio, quinolidinylthio, isoquinolylthio, quinolylthio, phtharazinylthio, naphthylidinylthio, quinolanylthio, quinazolinylthio, cinnolinylthio, pteridinylthio, carbazolylthio, phenanthridinylthio, acridinylthio, dibenzofuranylthio, benzoimidazolylthio, benzoisoxazolylthio, benzooxazolylthio, benzooxadiazolylthio, benzoisothiazolylthio, benzothiazolylthio, benzofurylthio, benzothienylthio, dibenzothienylthio, and benzodioxolylthio etc. are exemplified. Preferably furylthio, thienylthio, imidazolylthio, pyrazolylthio, isothiazolylthio, isoxazolylthio, oxazolylthio, thiazolylthio, pyridylthio, pyrazinylthio, pyrimidinylthio, and pyridazinylthio, etc.

The term “heteroarylsulfinyl” includes a group in which sulfinyl is substituted with one “heteroaryl” as described herein. Examples include pyrrolylsulfinyl, furylsulfinyl, thienylsulfinyl, imidazolylsulfinyl, pyrazolylsulfinyl, isothiazolylsulfinyl, isoxazolylsulfinyl, oxazolylsulfinyl, thiazolylsulfinyl, pyridylsulfinyl, pyrazinylsulfinyl, pyrimidinylsulfinyl, pyridazinylsulfinyl, tetrazolylsulfinyl, oxadiazolylsulfinyl, thiadiazolylsulfinyl, indolidinylsulfinyl, isoindolylsulfinyl, indolylsulfinyl, indazolylsulfinyl, purinylsulfinyl, quinolidinylsulfinyl, isoquinolylsulfinyl, quinolylsulfinyl, phtharazinylsulfinyl, naphthylidinylsulfinyl, quinolanylsulfinyl, quinazolinylsulfinyl, cinnolinylsulfinyl, pteridinylsulfinyl, carbazolylsulfinyl, phenanthridinylsulfinyl, acridinylsulfinyl, dibenzofuranylsulfinyl, benzoimidazolylsulfinyl, benzoisoxazolylsulfinyl, benzooxazolylsulfinyl, benzooxadiazolylsulfinyl, benzoisothiazolylsulfinyl, benzothiazolylsulfinyl, benzofurylsulfinyl, benzothienylsulfinyl, dibenzothienylsulfinyl, and benzodioxolylsulfinyl etc. are exemplified. Preferably furylsulfinyl, thienylsulfinyl, imidazolylsulfinyl, pyrazolylsulfinyl, isothiazolylsulfinyl, isoxazolylsulfinyl, oxazolylsulfinyl, thiazolylsulfinyl, pyridylsulfinyl, pyrazinylsulfinyl, pyrimidinylsulfinyl, and pyridazinylsulfinyl, etc.

The term “heteroarylsulfonyl” includes a group in which sulfonyl is substituted with one “heteroaryl” as described herein. Examples include pyrrolylsulfonyl, furylsulfonyl, thienylsulfonyl, imidazolylsulfonyl, pyrazolylsulfonyl, isothiazolylsulfonyl, isoxazolylsulfonyl, oxazolylsulfonyl, thiazolylsulfonyl, pyridylsulfonyl, pyrazinylsulfonyl, pyrimidinylsulfonyl, pyridazinylsulfonyl, tetrazolylsulfonyl, oxadiazolylsulfonyl, thiadiazolylsulfonyl, indolizinylsulfonyl, isoindolylsulfonyl, indolylsulfonyl, indazolylsulfonyl, purinylsulfonyl, quinolidinylsulfonyl, isoquinolylsulfonyl, quinolylsulfonyl, phtharazinylsulfonyl, naphthilidinylsulfonyl, quinolanylsulfonyl, quinazolinylsulfonyl, cinnolinyl sulfonyl, pteridinyl sulfonyl, carbazolylsulfonyl, phenanthridinylsulfonyl, acridinylsulfonyl, dibenzofuranylsulfonyl, benzoimidazolylsulfonyl, benzoisoxazolylsulfonyl, benzooxazolylsulfonyl, benzooxadiazolylsulfonyl, benzoisothiazolylsulfonyl, benzothiazolylsulfonyl, benzofurylsulfonyl, benzothienylsulfonyl, dibenzothienylsulfonyl, and benzodioxolylsulfonyl, ctc. Furylsulfonyl, thienylsulfonyl, imidazolylsulfonyl, pyrazolylsulfonyl, isothiazolylsulfonyl, isoxazolylsulfonyl, oxazolylsulfonyl, thiazolylsulfonyl, pyridylsulfonyl, pyrazinylsulfonyl, pyrimidinylsulfonyl, and pyridazinylsulfonyl are preferred.

The term “heteroarylsulfonyloxy” includes a group in which an oxygen atom is substituted with one “heteroarylsulfonyl” as described herein. Examples include pyrrolylsulfonyloxy, furylsulfonyloxy, thienylsulfonyloxy, imidazolylsulfonyloxy, pyrazolylsulfonyloxy, isothiazolylsulfonyloxy, isoxazolylsulfonyloxy, oxazolylsulfonyloxy, thiazolylsulfonyloxy, pyridylsulfonyloxy, pyrazinylsulfonyloxy, pyrimidinylsulfonyloxy, pyridazinylsulfonyloxy, tetrazolylsulfonyloxy, oxadiazolylsulfonyloxy, thiadiazolylsulfonyloxy, indolizinylsulfonyloxy, isoindolylsulfonyloxy, indolylsulfonyloxy, indazolylsulfonyloxy, purinylsulfonyloxy, quinolidinylsulfonyloxy, isoquinolylsulfonyloxy, quinolylsulfonyloxy, phtharazinylsulfonyloxy, naphthilidinylsulfonyloxy, quinolanyl sulfonyloxy, quinazolinylsulfonyloxy, cinnolinylsulfonyloxy, pteridinylsulfonyloxy, carbazolylsulfonyloxy, phenanthridinylsulfonyloxy, acridinylsulfonyloxy, dibenzofuranylsulfonyloxy, benzoimidazolylsulfonyloxy, benzoisoxazolylsulfonyloxy, benzooxazolylsulfonyloxy, benzooxadiazolylsulfonyloxy, benzoisothiazolylsulfonyloxy, benzothiazolylsulfonyloxy, benzofuryl sulfonyloxy, benzothienylsulfonyloxy, dibenzothienylsulfonyloxy, and benzodioxolylsulfonyloxy etc. are exemplified. Preferably, furylsulfonyloxy, thienylsulfonyloxy, imidazolylsulfonyloxy, pyrazolylsulfonyloxy, isothiazolylsulfonyloxy, isoxazolylsulfonyloxy, oxazolylsulfonyloxy, thiazolylsulfonyloxy, pyridylsulfonyloxy, pyrazinylsulfonyloxy, pyrimidinylsulfonyloxy, and pyridazinylsulfonyloxy, etc.

The term “aromatic carbocyclic ring” includes an aromatic monocyclic or aromatic fused carbocyclic ring. Examples include a benzene ring, a naphthalene ring, and an anthracene ring. A benzene ring is preferred.

The term “aromatic heterocyclic ring” includes an aromatic monocyclic or aromatic fused heterocyclic ring. Examples include a pyrrole ring, a furan ring, a thiophen ring, a pyrazole ring, an imidazole ring, an isothiazole ring, an isoxazole ring, an oxazole ring, a thiazole ring, a pyrazine ring, a pyrimidine ring, a pyridazine ring, a tetrazole ring, an oxadiazole ring, a thiadiazole ring, an indolizine ring, an isoindole ring, an indole ring, an indazole ring, a purine ring, a quinolidine ring, an isoquinoline ring, a quinoline ring, a phtharazine ring, a naphthyridine ring, a quinolane ring, a quinazoline ring, a cinnoline ring, a pteridine ring, a carbazole ring, a phenanthridine ring, an acridine ring, a dibenzofuran ring, a benzimidazole ring, a benzisoxazole ring, a benzoxazole ring, a benzoxadiazole ring, a benzisothiazole ring, a benzothiazole ring, a benzofuran ring, a benzothiophene ring, a dibenzothiophene ring, and a benzodixolane ring are exemplified. Preferably a pyridine ring, a furan ring, and a thiophen ring are exemplified.

The term “C1-C6 alkylene” includes a straight or branched alkylene group having one to six carbon atom(s). Examples include —CH₂—, —CH(CH₃)—, —C(CH₃)₂—, —CH₂CH₂—, —CH(CH₃)CH₂—, —C(CH₃)2CH₂—, —CH₂CH₂CH₂—, —CH₂CH₂CH₂CH₂—, —CH₂CH₂CH₂CH₂CH₂—, and —CH₂CH₂CH₂CH₂CH₂CH₂—. Preferred are —CH₂—, —CH₂CH₂—, —CH₂CH₂CH₂—, and —CH₂CH₂CH₂CH—.

The term “alkylene optionally containing one or two heteroatom(s)” of “optionally substituted alkylene optionally containing one or two heteroatom(s)” includes a straight or branched alkylene group having one to six carbon atoms, optionally containing one or two heteroatom(s) which may be substituted with “alkyl” as described herein. Examples include —CH₂—, —CH(CH₃)-, —C(CH₃)₂—, —CH₂CH₂—, CH₂CH₂CH₂—, —CH₂CH₂CH₂CH₂—, —CH₂CH₂CH₂CH₂CH₂—, —CH₂CH₂CH₂CH₂CH₂CH₂—, —CH₂₀-, —OCH₂—, —CH₂CH₂O—, —OCH₂CH₂—, —CH₂S—, —SCH₂—, —CH₂CH₂S—, —SCH₂CH₂—, —CH₂CH₂OCH₂CH₂—, —OCH₂CH₂O—, —OCH₂O—, —NHCH₂—, —N(CH₃)CH₂—, —N⁺(CH₃)₂CH₂—, —NHCH₂CH₂CH₂—, and —N(CH₃)CH₂CH₂CH₂—, etc. Preferred are —CH₂—, —CH₂CH₂—, —CH₂CH₂CH₂—, —CH₂CH₂CH₂CH₂—, —OCH₂CH₂O—, —OCH₂O—, and —N(CH₃)CH₂CH₂CH₂—.

The term “alkenylene optionally containing one or two heteroatom(s)” of “optionally substituted alkenylene optionally containing one or two heteroatom(s)” includes a straight or branched alkenylene group having two to six carbon atoms, optionally containing one or two heteroatom(s) which may be substituted with “alkyl” as described herein. Examples include —CH=CHCH═CH—, —CH═CHO—, —OCH═CH—, —CH═CHS—, —SCH═CH—, —CH=CHNH—, —NHCH═CH—, —CH═CH—CH═N—, and —N═CH—CH═CH—, Preferred are, —CH═CHCH═CH—, —CH═CHCH=N—, and —N═CHCH=CH—.

The term “alkynylene optionally containing one or two heteroatom(s)” includes a straight or branched alkynylene group having two to six carbon atoms, optionally containing one or two heteroatom(s) which may be substituted with “alkyl” as described herein. Examples include —C≡CCH₂—, —CH₂C≡CCH₂—, —CH₂C≡CCH₂O—, —OCH₂C≡CH—, —CH₂C≡CCH₂S—, —SCH₂C≡CH—, —CH₂C≡CCH₂NH—, —NHCH₂C≡CH—, —CH₂C≡CCH₂N(CH₃)—, and —N(CH₃)CH₂C≡CH—. Especially, —CH₂C≡CCH₂—, and —OCH₂C≡CH— are preferred.

The term “3- to 8-membered nitrogen-containing non-aromatic heterocyclic ring” includes a ring of any of the formulas described as such in U.S. Patent 8,143,285, which is incorporated herein by reference in its entirety.

The term “3- to 8-nitrogen-containing aromatic heterocyclic ring” includes a 3- to 8-membered aromatic heterocyclic ring containing one or more of nitrogen atom(s), and further optionally an oxygen atom and/or sulfur atom in the ring. Examples include pyrrolyl (e.g., 1-pyrrolyl, 2-pyrrolyl, 3-pyrrolyl), imidazolyl (e.g., 2-imidazolyl, 4-imidazolyl), pyrazolyl (e.g., 1-pyrazolyl, 3-pyrazolyl), isothiazolyl (e.g., 3-isothiazolyl), isoxazolyl (e.g., 3-isoxazolyl), oxazolyl (e.g., 2-oxazolyl), thiazolyl (e.g., 2-thiazolyl), pyridyl (e.g., 2-pyridyl, 3-pyridyl, 4-pyridyl), pyrazinyl (e.g., 2-pyrazinyl), pyrimidinyl (e.g., 2-pyrimidinyl, 4-pyrimidinyl), pyridazinyl (e.g., 3-pyridazinyl), tetrazolyl (e.g., 1H-tetrazolyl), oxadiazolyl (e.g., 1,3,4-oxadiazolyl), and thiadiazolyl (e.g., 1,3,4-thiadiazolyl).

The term “4- to 8-membered nitrogen-containing heterocyclic ring containing one or two nitrogen atom(s)” means a ring of any of the formulas described as such in U.S. Patent 8,143,285, which is incorporated herein by reference in its entirety.

The term “oxo” refers to an ═O group.

“Optionally substituted” is used interchangeably herein with “substituted or unsubstituted.”

In the present specification, examples of substituents in “optionally substituted alkyl,” “optionally substituted alkyloxy,” “optionally substituted alkylthio,” “optionally substituted alkylsulfinyl,” “optionally substituted alkylsulfonyl,” “optionally substituted alkylsulfonyloxy,” and “the” include cycloalkyl, alkylene optionally containing one or two heteroatom(s), hydroxyl, oxo, alkyloxy optionally substituted with a substituent group A at one to three position(s), thiol, alkylthio, halogen, nitro, cyano, carboxyl, sulfino (—SO₂H), alkyloxycarbonyl, optionally substituted amino, optionally substituted carbamoyl, acyl, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), an optionally substituted non-aromatic heterocyclic ring group (e.g., morpholinyl, pyrrolidinyl, piperazinyl) which may be substituted with a substituent group C at one to three position(s), aryloxy (e.g., phenyloxy) optionally substituted with a substituent group B at one to three position(s), alkylsulfonyl, and the like. The above-referenced “optionally substituted” moieties can be substituted with one to three of the above-referenced substituent(s) at any possible position.

In the present specification, examples of substituents in “optionally substituted alkenyl,” “optionally substituted alkynyl,” “optionally substituted alkenyloxy,” “optionally substituted alkynyloxy,” “optionally substituted alkenylthio,” “optionally substituted alkynylthio,” “optionally substituted alkenyloxycarbonyl,” “optionally substituted alkynyloxycarbonyl,” “optionally substituted cycloalkyl,” “optionally substituted cycloalkenyl,” “optionally substituted cycloalkyloxy, “optionally substituted cycloalkenyloxy,” “optionally substituted cycloalkylthio,” “optionally substituted cycloalkenylthio,” “optionally substituted cycloalkylsulfinyl,” “optionally substituted cycloalkenylsulfinyl,” “optionally substituted cycloalkylsulfonyl,” “optionally substituted cycloalkenylsulfonyl,” “optionally substituted cycloalkylsulfonyloxy,” “optionally substituted cycloalkenylsulfonyloxy,” “optionally substituted alkenyloxycarbonyl,” “optionally substituted alkylene,” “optionally substituted C1 -C6 alkylene,” “optionally substituted alkylene optionally containing one or two heteroatom(s),” “optionally substituted alkenylene,” “optionally substituted alkenylene optionally containing one or two heteroatom(s),” “optionally substituted alkynylene,” and “optionally substituted alkynylene optionally containing one or two heteroatom(s)” include alkyl (such as dialkyl) optionally substituted with a substituent group D at one to three position(s), cycloalkyl, hydroxyl, oxo, alkyloxy optionally substituted with a substituent group A at one to three position(s), thiol, alkylthio, halogen, nitro, cyano, carboxyl, sulfino, alkyloxycarbonyl, optionally substituted amino, optionally substituted carbamoyl, acyl, acyloxy, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), non-aromatic heterocyclic group (e.g., morpholinyl, pyrrolidinyl, piperazinyl) optionally substituted with a substituent group C at one to three position(s), aryloxy (e.g., phenyloxy) optionally substituted with a substituent group C at one to three position(s), alkylsulfonyl, and the like. The above-referenced “optionally substituted” moieties can be substituted with one or more of the above-referenced substituent(s) at any possible position.

In the present specification, examples of substituents in “optionally substituted aryl,” “optionally substituted phenoxy,” “optionally substituted aryloxy,” “optionally substituted phenylthio,” “optionally substituted arylthio,” “optionally substituted arylsulfinyl,” “optionally substituted arylsulfonyl,” “optionally substituted arylsulfonyloxy,” “optionally substituted heteroaryl,” “optionally substituted heteroaryloxy,” “optionally substituted heteroarylthio,” “optionally substituted heteroarylsulfinyl,” “optionally substituted heteroarylsulfonyl,” “optionally substituted heteroarylsulfonyloxy,” “optionally substituted non-aromatic heterocyclic group,” “optionally substituted C6 arene-1,4-diamine-N¹,N⁴-diyl,” and substituted C6 arene-1,4-diamine-N¹,N⁴-diyl,” include alkyl optionally substituted with a substituent group D at one to three position(s), cycloalkyl, alkenyl, alkynyl, hydroxyl, alkyloxy optionally substituted with a substituent group A at one to three position(s), aryloxy (e.g., phenoxy) optionally substituted with a substituent group B at one to three position(s), thiol, alkylthio, halogen, nitro, cyano, carboxyl, sulfino, alkyloxycarbonyl, acyl, alkylsulfonyl, optionally substituted amino, optionally substituted carbamoyl, aryl (e.g., phenyl) optionally substituted with a substituent group B at one to three position(s), heteroaryl (e.g., pyridyl, furyl, thicnyl, imidazolyl, oxazolyl, thiazolyl, pyrazolyl) optionally substituted with a substituent group C at one to three position(s), non-aromatic heterocyclic group (e.g., morpholinyl, pyrrolidinyl, piperazinyl) optionally substituted with a substituent group C at one to three position(s), and the like. The above-referenced “optionally substituted” moieties can be substituted with one or more of the above-referenced substituent(s) at any possible position.

Substituent group A is comprised of halogen and phenyl optionally substituted with one to three substituent(s) selected from the Substituent group B.

Substituent group B is comprised of halogen, alkyl, alkyloxy, cyano, and nitro.

Substituent group C is comprised of halogen and alkyl.

Substituent group D is comprised of halogen and alkyloxy.

“- - - ” between adjacent atoms indicates a bond that is present or absent depending on the valency of the adjacent atoms in a given specified structural context. The bond may comprise localized electrons between the adjacent atoms or delocalized electrons depending on the given specified structural context.

“Exactly one” means one and only one.

It is preferred that the ring formed by A¹⁰, A¹², A¹³, A¹⁴, and A¹⁵ of Yl includes no more than three constituent ring heteroatoms. In some versions, the ring formed by A¹⁰, A¹², A¹³, A¹⁴, and A¹⁵ of Y¹ includes no more than two constituent ring heteroatoms. In some versions, the ring formed by A¹⁰, A¹², A¹³, A¹⁴, and A¹⁵ of Y¹ includes no more than one constituent ring heteroatom.

It is preferred that the ring formed by A¹⁵, A¹⁶, A¹⁷, and A¹⁸ of Y² includes no more than three constituent ring heteroatoms. In some versions, the ring formed by A¹⁵, A¹⁶, A¹⁷, and A¹⁸ of Y² includes no more than two constituent ring heteroatoms. In some versions, the ring formed by A¹⁵, A¹⁶, A¹⁷, and A¹⁸ of Y² includes no more than one constituent ring heteroatom.

It is preferred that the ring formed by A¹, A², A³, A⁴, AS, and A⁶ includes no more than three constituent ring heteroatoms. In some versions, the ring formed by A¹, A², A³, A⁴, A⁵, and A⁶ includes no more than two constituent ring heteroatoms. In some versions, the ring formed by A¹, A², A³, A⁴, A⁵, and A⁶ includes no more than one constituent ring heteroatom.

It is preferred that the ring formed by A⁵, A⁶ A⁷, A⁸, and A⁹ includes no more than three constituent ring heteroatoms. In some versions, the ring formed by A⁵, A⁶ A⁷, A⁸, and A⁹ includes no more than two constituent ring heteroatoms. In some versions, the ring formed by A⁵, A⁶ A⁷, A⁸, and A⁹ includes no more than one constituent ring heteroatom.

It is preferred that the ring formed by A¹⁹, A²⁰, A²¹, A²², and A²³ of Z includes no more than three constituent ring heteroatoms. In some versions, the ring formed by A¹⁹, A²⁰, A²¹, A²², and A²³ of Z includes no more than two constituent ring heteroatoms. In some versions, the ring formed by A¹⁹, A²⁰, A²¹, A²², and A²³ of Z includes no more than one constituent ring heteroatom.

In some versions, at least one substituent in any pair of substituents of constituent ring atoms of A¹, A², A³, A⁴, A⁵, A⁶, A⁷, A⁸, A⁹, A¹⁰, A¹¹, A¹², A¹³, A¹⁴, A¹⁵, A¹⁶, A¹⁷, A¹⁸, A¹⁹, A²⁰, A²¹, A²², and A²³, unless explicitly specified otherwise, is a non-cyclic moiety. In some versions, at least one substituent in any pair of substituents of constituent ring atoms of A¹, A², A³, A⁴, A⁵, A⁶, A⁷, A⁸, A⁹, A¹⁰, A¹¹, A¹², A¹³, A¹⁴, A¹⁵, A¹⁶, A¹⁷, A¹⁸, A¹⁹, A²⁰, A²¹, A²², and A²³, unless explicitly specified otherwise, is independently hydrogen, halogen, or optionally substituted C1 -C6 alkyl. In some versions, at least one substituent in any pair of substituents of constituent ring atoms of A¹, A², A³, A⁴, A⁵, A⁶, A⁷, A⁸, A⁹, A¹⁰, A¹¹, A¹², A¹³, A¹⁴, A¹⁵, A¹⁶, A¹⁷, A¹⁸, A¹⁹, A²⁰, A²¹, A²², and A²³, unless explicitly specified otherwise, is independently hydrogen or halogen. In some versions, at least one substituent in any pair of substituents of constituent ring atoms of A¹, A², A³, A⁴, A⁵, A⁶, A⁷, A⁸, A⁹, A¹⁰, A¹¹, A¹², A¹³, A¹⁴, A¹⁵, A¹⁶, A¹⁷, A¹⁸, A¹⁹, A²⁰, A²¹, A²², and A²³, unless explicitly specified otherwise, is independently hydrogen. “Vicinal” in this context refers to any two substituents bonded to adjacent constituent ring atoms.

In the course of the methods of the present invention, an effective amount of a compound of the invention can be administered to an animal, including mammals and humans, in many ways. While in the preferred embodiment, the compounds of the invention are administered orally, parenterally, or topically, other forms of administration such as through medical compounds or aerosols are also contemplated. “Effective amount” is used herein to refer to an amount effective to treat a given condition or disease or a given type of condition or disease.

For oral administration, the effective amount of compounds may be administered in, for example, a solid, semi-solid, liquid, or gas state. Specific examples include tablet, capsule, powder, granule, solution, suspension, syrup, and elixir agents. However, the compounds are not limited to these forms.

To formulate the compounds of the invention into tablets, capsules, powders, granules, solutions, or suspensions, the compound is preferably mixed with a binder, a disintegrating agent and/or a lubricant. If necessary, the resultant composition may be mixed with a diluent, a buffer, an infiltrating agent, a preservative and/or a flavor, using known methods. Examples of the binder include crystalline cellulose, cellulose derivatives, cornstarch, cyclodextrins, and gelatin. Examples of the disintegrating agent include cornstarch, potato starch, and sodium carboxymethylcellulose. Examples of the lubricant include talc and magnesium stearate. Further, additives, which have been conventionally used, such as lactose and mannitol, may also be used.

For parenteral administration, the compounds of the present invention may be administered rectally or by injection. For rectal administration, a suppository may be used. The suppository may be prepared by mixing the compounds of the present invention with a pharmaceutically suitable excipient that melts at body temperature but remains solid at room temperature. Examples include but are not limited to cacao butter, carbon wax, and polyethylene glycol. The resulting composition may be molded into any desired form using methods known to the field.

For administration by injection, the compounds of the present invention may be injected hypodermically, intracutaneously, intravenously, or intramuscularly. Medicinal drugs for such injection may be prepared by dissolving, suspending or emulsifying the compounds of the invention into an aqueous or non-aqueous solvent such as vegetable oil, glyceride of synthetic resin acid, ester of higher fatty acid, or propylene glycol by a known method. If desired, additives such as a solubilizing agent, an osmoregulating agent, an emulsifier, a stabilizer, or a preservative, which has been conventionally used may also be added. While not required, it is preferred that the composition be sterile or sterilized.

To formulate the compounds of the invention into suspensions, syrups, or elixirs, a pharmaceutically suitable solvent may be used. Included among these is the non-limiting example of water.

For topical administration, topical formulations can be in a form of gel, cream, lotion, liquid, emulsion, ointment, spray, solution, suspension, and patches. Inactive ingredients in the topical formulations for example include, but not limited to, lauryl lactate (emollient/permeation enhancer), diethylene glycol monocthylether (emollient/permeation enhancer), DMSO (solubility enhancer), silicone elastomer (rheology/texture modifier), caprylic/capric triglyceride, (emollient), octisalate, (emollient/UV filter), silicone fluid (emollient/diluent), squalene (emollient), sunflower oil (emollient), and silicone dioxide (thickening agent).

The compounds of the invention may also be used together with an additional compound having other pharmaceutically suitable activity to prepare a medicinal drug. A drug, either containing a compound of the invention as a stand-alone compound or as part of a composition, may be used in the treatment of subjects in need thereof.

The compounds of the invention may also be administered in the form of an aerosol or inhalant prepared by charging the compounds in the form of a liquid or fine powder, together with a gaseous or liquid spraying agent and, if necessary, a known auxiliary agent such as an inflating agent, into a non-pressurized container such as an aerosol container or a nebulizer. A pressurized gas of, for example, dichlorofluoromethane, propane or nitrogen may be used as the spraying agent.

The compounds of the invention may be administered to an animal, including mammals and humans, in need thereof as a pharmaceutical composition, such as tablets, capsules, solutions, or emulsions.

The compounds of the invention may also be administered to an animal in need thereof as a nutritional additive, either as a food or nutraceutical supplement.

The term “treating” refers to the reduction, by any degree, of a condition or any aspect, complication, or symptom thereof. Examples include reducing the severity of the condition, reducing the number of symptoms or complications of the condition, eliminating a particular symptom or complication of the condition, reducing the severity of one or more symptoms or complications of the condition, or eliciting any other change in the condition of the patient that improves the therapeutic outcome.

The term “preventing” refers to the prophylactic reduction, by any degree, of a condition or any aspect, complication or symptom thereof. Examples include prophylactically reducing the severity of the condition, prophylactically reducing the number of symptoms or complications of the condition, prophylactically eliminating a particular symptom or complication of the condition, prophylactically reducing the severity of one or more symptoms or complications of the condition, or prophylactically eliciting any other change in the condition of the patient that improves the therapeutic outcome.

The compounds described in this invention are preferably used and/or administered in the form of a composition. Suitable compositions are, preferably, a pharmaceutical composition, a foodstuff, or a food supplement. These compositions provide a convenient form in which to deliver the compounds. Compositions of the invention may comprise an antioxidant in an amount effective to increase the stability of the compounds with respect to oxidation or solubility.

The amount of compound that is administered in the method of the invention or that is for administration in the use of the invention is any suitable amount. Examples include from 1 ng/kg body weight to 20 g/kg body weight, such as from 1 μg/kg body weight to 1 g/kg body weight or from 1 mg/kg body weight to 100 mg/kg body weight of compound per day. Suitable compositions can be formulated accordingly. Those of skill in the art of dosing of biologically active agents will be able to develop particular dosing regimens for various subjects based on known and well understood parameters.

A preferred composition according to the invention is a pharmaceutical composition, such as in the form of tablets, pills, capsules, caplets, multiparticulates (including granules, beads, pellets and micro-encapsulated particles), powders, elixirs, syrups, suspensions, and solutions. Pharmaceutical compositions will typically comprise a pharmaceutically acceptable diluent or carrier. Pharmaceutical compositions are preferably adapted for administration parenterally or orally. Orally administrable compositions may be in solid or liquid form and may take the form of tablets, powders, suspensions, and syrups, among other things. Optionally, the compositions comprise one or more flavoring and/or coloring agents. In general, therapeutic and nutritional compositions may comprise any substance that does not significantly interfere with the action of the compounds on the subject.

Pharmaceutically acceptable carriers suitable for use in such compositions are well known in the art of pharmacy. The compositions of the invention may contain 0.01-99% by weight of the compounds of the invention. The compositions of the invention are generally prepared in unit dosage form. Examples of unit dosages of the compounds of the invention include from 0.1 mg to 2000 mg, such as 50 mg to 1000 mg. The excipients used in the preparation of these compositions can include any excipients known in the art.

Further examples of product forms for the composition are food supplements, such as in the form of a soft gel or a hard capsule comprising an encapsulating material selected from the group consisting of gelatin, starch, modified starch, starch derivatives such as glucose, sucrose, lactose, and fructose. The encapsulating material may optionally contain cross-linking or polymerizing agents, stabilizers, antioxidants, light absorbing agents for protecting light-sensitive fills, preservatives, and the like.

In general, the term “carrier” represents a composition with which the compounds described may be mixed, be it a pharmaceutical carrier, foodstuff, nutritional supplement, or dietary aid. The materials described above may be considered carriers for the purposes of the invention. In certain embodiments of the invention, the carrier has little to no biological activity on the compounds of the invention.

Dose: The methods of the present invention can comprise administering a therapeutically effective amount of compound to an animal in need thereof. The effective amount of compound depends on the form of the compound administered, the duration of the administration, the route of administration (e.g., oral or parenteral), the age of the animal, and the condition of the animal, including mammals and humans. Exemplary amounts range from 1 ng/kg/day to 20 g/kg/day, such as 50 μg/kg/day to 5 g/kg/day or 1 to 100 mg/kg/day. The effective amount of compound is most effective in treating or preventing the condition when administered for periods ranging from about 1 to 1000 days or more, such as from 7 to 300 days or from 30 to 90 days. The effective amount of compound may be continued beyond these periods for maintenance of beneficial responses in chronic diseases.

When the effective amount of the compound of the present invention is administered in a nutritional, therapeutic, medical, or veterinary composition, an exemplary dose ranges from about 0.01 to 2.0% wt/wt to the food or nutraceutical product.

In general, the present invention relates to inhibition of inflammation systemically, wherein relevant components include the pancreas, spleen, lung, heart, cardiovascular system, central nervous system, joints, liver, kidneys, immune system, or GI tract. Relevant components in the GI tract include the esophagus, stomach, small intestine, cecum, large intestine, and rectum. The effects result from the exposure of various cells types in the body that induce a biological effect to a compound of the invention. The cells may include those from pulmonary or skin tissues, immune cells (i.e., macrophages, monocytes, cosinophils, dendritic cells, neutrophils, lymphocytes), pancreatic islet cells, endothelial cells, neurons, or epithelial cells, among others.

When practiced, the methods of the invention can be by way of administering the compounds to a subject via any acceptable administration route using any acceptable form, as is described above, and allowing the body of the subject to distribute the compounds to the target tissues and cells through natural processes. As is described above, administering can likewise be by direct injection to a site (e.g., organ, tissue) containing a target cell (i.e., a cell to be treated).

The amount to be administered will vary depending on the subject, stage of disease or disorder, age of the subject, general health of the subject, and various other parameters known and routinely taken into consideration by those of skill in the medical arts. As a general matter, a sufficient amount of compound will be administered in order to make a detectable change in the amount of inflammation systemically or in any particular tissue or site in the body. Reduction of inflammation may be related to amount of pain experienced by the subject, insulin, anti-nuclear antigen antibodies, TNFα, or C-reactive protein levels in the blood, the percent of cosinophils or regulatory T-cells in the blood, sputum or target tissue, or concentration of calprotectin in feces.

The methods of the present invention can provide treatments for reducing inflammation by affecting the metabolism of immune cells. The methods can reduce inflammation systemically (i.e., throughout the subject's body) or locally (e.g., at the site of administration or the site of inflammatory cells, including but not limited to T cells and macrophages). In treating or preventing inflammation through immunometabolism, one effect that may be observed is a shift in the metabolism of glucose. In particular, the shift may be from the production of lactate from pyruvate towards the entrance into the tricarboxylic acid cycle that is tied with immunoinflammatory actions. More specifically, this shift in metabolism can be associated with an increase in the proportion of CD4+CD25+FOXP3+or other regulatory CD4+ T-cells relative to effector CD4+ T-cells such as Th2, IL17+ Th17 or IFNγ+ Th1 effector cells. Another observed effect may be decreased cellular proliferation resulting from the combination of decreased anaerobic metabolism and increased immune checkpoint pathways. Another effect of shifts in metabolism triggered therapeutically may be decreased expression of inflammatory chemokines such as MCP-1, IL-8, or CXCL9 resulting from altered processing and storage of fatty acids. The methods can thus also be considered methods of affecting or altering the immune response of a subject to whom the therapy is administered, thereby intercepting inflammation, discase and pathology.

The methods of the present invention can provide methods of reducing inflammation by producing other effects. The methods can reduce inflammation systemically (i.e., throughout the subject's body) or locally (e.g., at the site of administration or the site of inflammatory cells, including but not limited to cosinophils, T cells and macrophages). In treating or preventing inflammation according to the methods of the present invention, one effect that may be seen is the decrease in the number of blood monocytes or macrophages and lymphocytes infiltrating a given tissue. Another may be the increase in regulatory immune cell populations, such as CD4⁺CD25⁺FoxP3⁺ regulatory T-cells, or an increase in regulatory properties of lymphocytes or macrophages (e.g., increased interleukin 4 (IL-4) or IL-10 or decreased TNF-α and IL-6). Another may be the decreased presence of inflammatory genes and/or adhesion molecules. The methods can thus also be considered methods of affecting or altering the immune response of a subject to whom the therapy is administered. The subject may have any condition in which the immunomodulation of T cells or downregulation of cellular adhesion molecules is a desired outcome.

The invention provides methods of treating allergic, inflammatory, or immune-mediated diseases with the compounds described herein. The inflammatory or immune-mediated disease can include any disease described in Dattatreya et al. 2011 and Shurin et al. 2007, among others.

The invention provides methods of treating autoimmune diseases, such as inflammatory autoimmune diseases, with the compounds described herein. Non-limiting examples of autoimmune diseases include inflammatory bowel disease (IBD) (e.g., Crohn's disease and ulcerative colitis), irritable bowel syndrome (IBS), lupus, systemic lupus erythematosus, rheumatoid arthritis, Sjogren's syndrome, systemic scleroderma, type 1 diabetes, psoriasis (including psoriatic arthritis), autoimmune encephalitis, multiple sclerosis, sarcoidosis, Guillain-Barre syndrome, Grave's disease, antiphospholipid syndrome and cancer-immunotherapy-induced autoimmune diseases, among others. Non-limiting examples of cancer-immunotherapy-induced autoimmune diseases include cancer immunotherapy-induced rheumatic diseases. Non-limiting examples of multiple sclerosis include relapsing-remitting multiple sclerosis, secondary progressive multiple sclerosis, and primary progressive multiple sclerosis. The invention also provides methods of treating inflammation associated with autoimmune diseases.

The compounds of the invention can be used to treat the symptoms in a subject diagnosed with systemic lupus erythematosus or to prevent the development of disease in a subject genetically predisposed to systemic lupus erythematosus. Symptoms and indications of lupus that may be treated with the invention include but are not limited to lupus nephritis, central nervous system inflammation, headaches, scleritis, optic neuritis, fevers, hardening of the arteries, coronary artery disease, joint pain and malar rash. The invention also provides a method of treating additional forms of lupus including cutaneous lupus (discoid), drug-induced lupus and neonatal lupus.

The compounds of the invention can be used to treat diabetes or conditions resulting therefrom. Exemplary types of diabetes include type 1 diabetes and type 2 diabetes. Exemplary diabetes conditions include diabetic nephropathy, diabetic retinopathy, chronic pain, neuropathy, deep vein thrombosis, or atherosclerosis.

The invention provides methods of treating chronic inflammatory diseases with the compounds described herein. Non-limiting examples of chronic inflammatory diseases includes metabolic syndrome, obesity, prediabetes, cardiovascular disease, type 2 diabetes, non-alcoholic fatty liver disease, non-alcoholic steatohepatitis, cirrhosis, asthma, allergies, chronic granulomatous disease, graft versus host disease, and tumor necrosis factor receptor associated periodic syndrome; muscle wasting, such as amyotrophic lateral sclerosis, Duchenne muscular dystrophy, scoliosis, and progressive muscular atrophy; and others.

The invention provides methods of treating other inflammatory diseases such as acute colonic diverticulitis and radiation-induced inflammation of the gastrointestinal tract with the compounds described herein. Non-limiting examples of radiation-induced inflammation of the gastrointestinal tract include radiation proctitis, radiation enteritis, and radiation proctosigmoiditis.

The invention provides methods of treating allergic diseases with the compounds described herein. Examples of allergic diseases include hay fever (seasonal allergies), sinusitis, asthma, eczema, hives, anaphylaxis.

The invention provides methods of treating chronic and/or inflammatory respiratory diseases with the compounds described herein. Non-limiting examples of chronic and/or inflammatory respiratory diseases include asthma, chronic obstructive pulmonary diseases, such as emphysema, pulmonary fibrosis, such as idiopathic pulmonary fibrosis, and granulomatous lung disease.

The invention provides methods of treating asthma with the compounds described herein. Exemplary forms of asthma treated with the compounds described herein include chronic asthma, acute asthma, allergic asthma, type 2 asthma, cosinophilic asthma, non-type 2 asthma, and neutrophilic asthma.

The invention provides methods of treating chronic and/or inflammatory central nervous diseases with the compounds described herein. Non-limiting examples of chronic and/or inflammatory central nervous diseases include Alzheimer's disease, Parkinson's disease, neuroinflammation resulting from stroke, traumatic brain injury, or spinal cord injury.

The invention provides methods of treating inflammatory or immune-mediated conditions of the skin. Exemplary inflammatory or immune-mediated conditions of the skin include psoriasis, cutaneous lupus erythematosus, dermatomyositis, pemphigoid, pemphigus, scleroderma, vasculitis, epidermolysis bullosa acquisita, vitiligo, lichen planus, scleritis, dermatitis, erythema nodosum, pyoderma gangrenosum, skin fissures, acne, enterocutaneous fistula, skin tags, canker sores, acrodermatitis enteropathica, pyoderma vegetans, leukocytoclastic vasculitis, anal fissures, Sweet's syndrome, rosacea, alopecia, keratoderma blennorrhagica, rosacea, cold sores, urticaria, actinic keratosis, carbuncle, cellulitis, ichthyosis vulgaris, skin infection, malar rash, photosensitivity, livedo reticularis, livedo reticularis, oral and nasal ulcers, purpura, mucositis, hemorrhoids, burn, and sunburn.

The invention provides methods of treating allergic disorders of the skin with the compounds described herein. Exemplary allergic disorders of the skin include dermatitis, such as atopic dermatitis (eczema) and contact dermatitis, hives (urticaria), and swelling (angioedema).

The invention provides methods of inhibiting inflammation or treating an allergic disease in the gastrointestinal tract with the compounds described herein, wherein relevant components of the gastrointestinal tract can include the stomach, small intestine, large intestine, and rectum. The allergic diseases of the gastrointestinal tract can include an cosinophilic gastrointestinal disorder. The cosinic gastrointestinal disorder can comprise cosinophilic gastroenteritis or cosinophilic esophagitis.

The invention provides methods of treating hypercosinophilic syndrome with the compounds described herein.

The invention provides methods of treating a granulomatous disorder with the compounds described herein. Exemplary granulomatous disorders include Churg-Strauss syndrome, berylliosis, and sarcoidosis, among others. See James DG. A clinicopathological classification of granulomatous disorders. Postgrad Med J. 2000;76(898):457-465. doi: 10.1136/pmj.76.898.457. The granulomatous disorder can be from infectious origin (e.g., tuberculosis), environmental exposure (e.g., beryllium exposures), or chronic disease (e.g., sarcoidosis), among other origins.

The invention provides methods of treating an infectious disease with the compounds described herein. Non-limiting examples of such infectious diseases include viral infections, bacterial infections, and fungal infections.

Non-limiting examples of viral infections include infections from viruses in the family adenoviridae, such as adenovirus; viruses in the family herpesviridae such as herpes simplex, type 1, herpes simplex, type 2, varicella-zoster virus, epstein-barr virus, human cytomegalovirus, human herpesvirus, and type 8; viruses in the family papillomaviridae such as human papillomavirus; viruses in the family polyomaviridae such as BK virus and JC virus; viruses in the family poxviridae such as smallpox; viruses in the familyhepadnaviridae such as hepatitis B virus; viruses in the family parvoviridae such as human bocavirus and parvovirus B19; viruses in the family astroviridae such as human astrovirus; viruses in the family caliciviridae such as norwalk virus; viruses in the family picornaviridae such as coxsackievirus, hepatitis A virus, poliovirus, and rhinovirus; viruses in the family coronaviridae such as acute respiratory syndrome virus; viruses in the family flaviviridae such as hepatitis C virus, yellow fever virus, dengue virus, and West Nile virus, viruses in the family togaviridae such as rubella virus; viruses in the family hepeviridae such as hepatitis E virus; viruses in the family retroviridae such as human immunodeficiency virus (HIV); viruses in the family orthomyxoviridae such as influenza virus; viruses in the family arenaviridae such as guanarito virus, junin virus, lassa virus, machupo virus, and sabiá virus; viruses in the family bunyaviridae such as Crimean-Congo hemorrhagic fever virus; viruses in the family filoviridae such as ebola virus and marburg virus; coronavirus (COVID-19); viruses in the family paramyxoviridae such as measles virus, mumps virus, parainfluenza virus, respiratory syncytial virus, human metapneumovirus, hendra virus, and nipah virus; viruses in the family rhabdoviridae such as rabies virus; unassigned viruses such as hepatitis D virus; and viruses in the family reoviridae such as rotavirus, orbivirus, coltivirus, and banna virus, among others.

Non-limiting examples of bacterial infections include infections with the bacteria described above, in addition to Bacillus anthracis, Bacillus cereus, Bordetella pertussis, Borrelia burgdorferi, Brucella abortus, Brucella canis, Brucella melitensis, Brucella suis Campylobacter jejuni Chlamydia pneumoniae, Chlamydia trachomatis, Chlamydophila psittaci, Clostridium botulinum, Clostridium difficile, Clostridium perfringens, Clostridium tetani, Corynebacterium diphtheriae, Enterococcus faecalis, Enterococcus faecium, Escherichia coli, Francisella tularensis, Haemophilus influenzae, Helicobacter pylori, Legionella pneumophila, Leptospira interrogans, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Rickettsia rickettsii, Salmonella typhi, Salmonella typhimurium, Shigella sonnei, Staphylococcus aureus, Staphylococcus epidermidis, Staphylococcus saprophyticus, Streptococcus agalactiae, Streptococcus pneumoniae, Streptococcus pyogenes, Treponema pallidum, Vibrio cholerae, Yersinia pestis, Yersinia enterocolitica, Yersinia pseudotuberculosis, and other species from the genera of the above-mentioned organisms.

Non-limiting examples of fungal infections include infection with fungi of the genus Aspergillus, such as Aspergillus fumigatus, which cause aspergillosis; fungi of the genus Blastomyces, such as Blastomyces dermatitidis, which cause blastomycosis; fungi of the genus Candida, such as Candida albicans, which cause candidiasis; fungi of the genus Coccidioides, which cause coccidioidomycosis (valley fever); fungi of the genus Cryptococcus, such as Cryptococcus neoformans and Cryptococcus gattii, which cause cryptococcosis; dermatophytes fungi, which cause ringworm; fungi that cause fungal keratitis, such as Fusarium species, Aspergillus species, and Candida species; fungi of the genus Histoplasma, such as Histoplasma capsulatum, which cause histoplasmosis; fungi of the order Mucorales, which cause mucormycosis; fungi of the genus Saccharomyces, such as Saccharomyces cerevisiae; fungi of the genus Pneumocystis, such as Pneumocystis jirovecii, which cause pneumocystis pneumonia; and fungi of the genus Sporothrix, such as Sporothrix schenckii, which cause sporotrichosis.

The invention also provides methods of treating hyperproliferative disorders with the compounds described herein. Hyperproliferative disorders include conditions involving uncontrolled growth of cells, such as cancers or conditions involving the growth of tumors, adenomas, or polyps. Non-limiting examples of hyperproliferative disorders include colorectal cancer, familial adenomatous polyposis (PAP), throat cancer, thyroid cancer, gastric cancer, cancers of the gastrointestinal tract, pancreatic cancer, Hodgkin lymphoma, non-Hodgkin lymphoma, acute myeloid leukemia, hepatocellular cancer, gastrointestinal stromal tumors, acute lymphoblastic leukemia, chronic myeloproliferative disorders, hypereosinophilic syndrome, mastocytosis, among others.

As used herein, “tetrahydropyrazolopyridine-analog” refers to compounds comprising a tetrahydropyrazolopyridine moiety or structural analogs thereof.

The depiction or definition of any moiety or compound provided herein encompasses any tautomer of the moiety or compound, unless the context clearly dictates otherwise.

The depiction or definition of any moiety or compound provided herein encompasses any salt of the moiety or compound, unless the context clearly dictates otherwise.

The elements, embodiments, versions, and method steps described herein can be used in any compatible combination whether explicitly described or not.

A¹¹ combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

A¹¹ patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Molecular Modeling

### Example 1. Molecular Modeling of NLRX1 Ligands

Using previously described ligands of NLRX1, including viral RNA and dietary lipids (punicic acid and docosahexaenoic acid), we determined the existence of two high-potential binding sites on the NLRX1 protein (Lu et al. 2015). These ligands were docked onto the published structure for the C terminus of NLRX1 (pdb: 3UN9) to establish important binding residues.

### Methods

Virtual Screening. To provide additional insights into preliminary scaffolds, ligand databases were docked onto the NLRX1 using AutoDock Vina at each of the two sites using cuboid search grid of size (58×40×40 angstrom) to provide predicted binding affinities and conformations of ligands. Binding affinity was normalized to molecular weight of the ligand. Top ligands were selected for further examination of binding pose.

Compound generation. From the identified residues and predicted biochemical interactions, structures were generated for high affinity NLRX1 ligands. Structures were generated and chemically optimized using WebMo. Structure files were generated in .pdb format and converted to .pdbqt format through calculation of charges by Gasteiger method. Structures were docked using AutoDock Vina to confirm binding affinity.

Analysis. Compounds were preliminarily ranked by lowest predicted binding affinity normalized to molecular weight representing the most favorable binding pose through a minimization of total intermolecular energy, total internal energy and torsional free energy. Compounds were then prioritized based on favorable distances to critical binding residues on NLRX1.

### Results

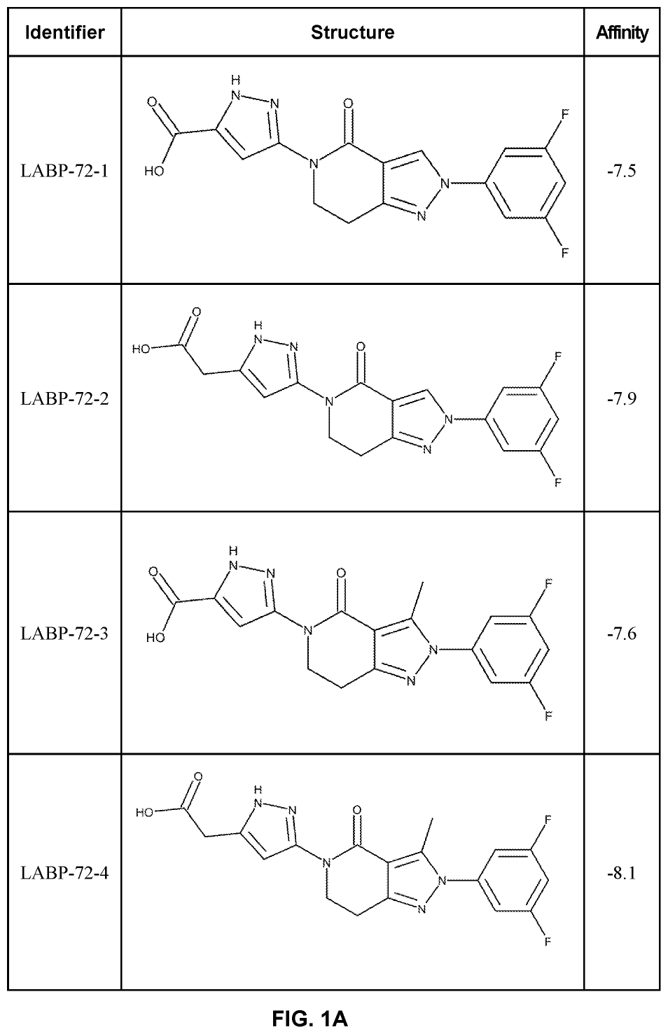

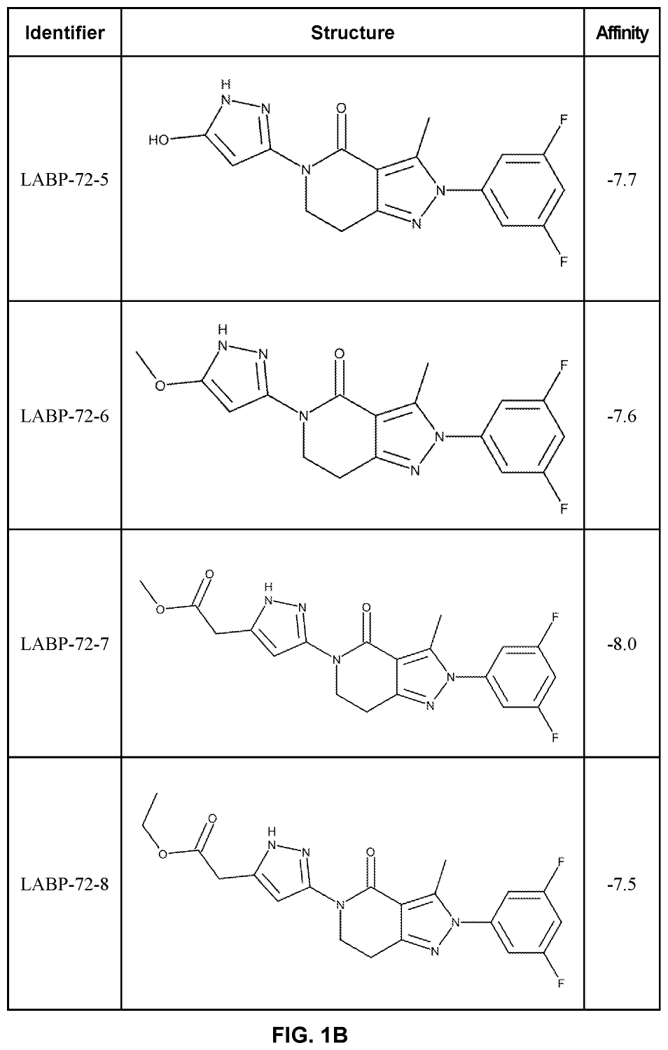

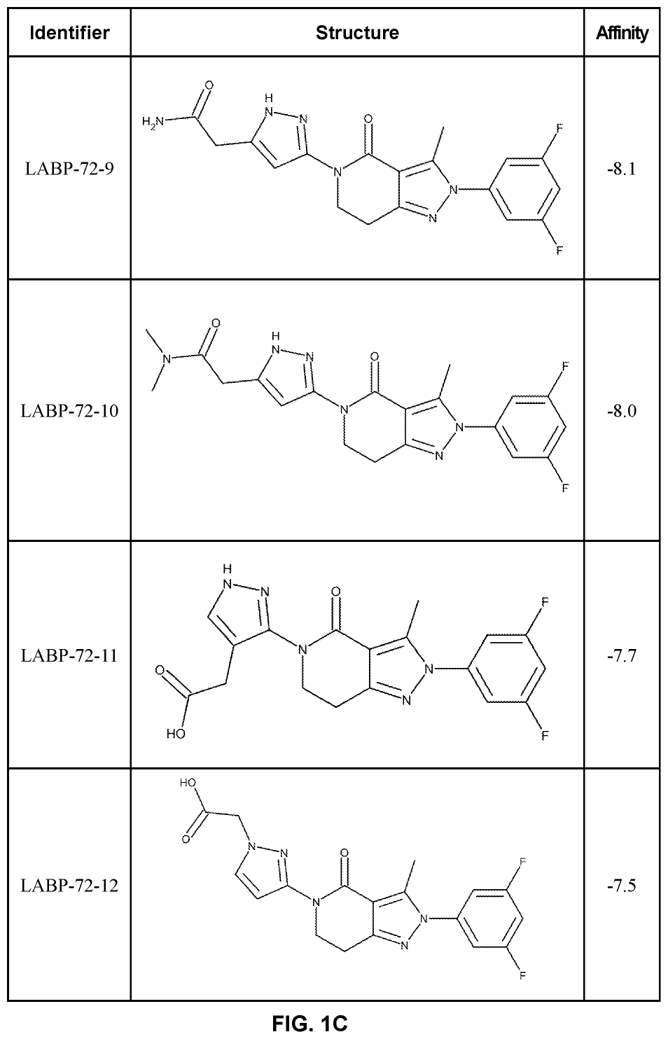

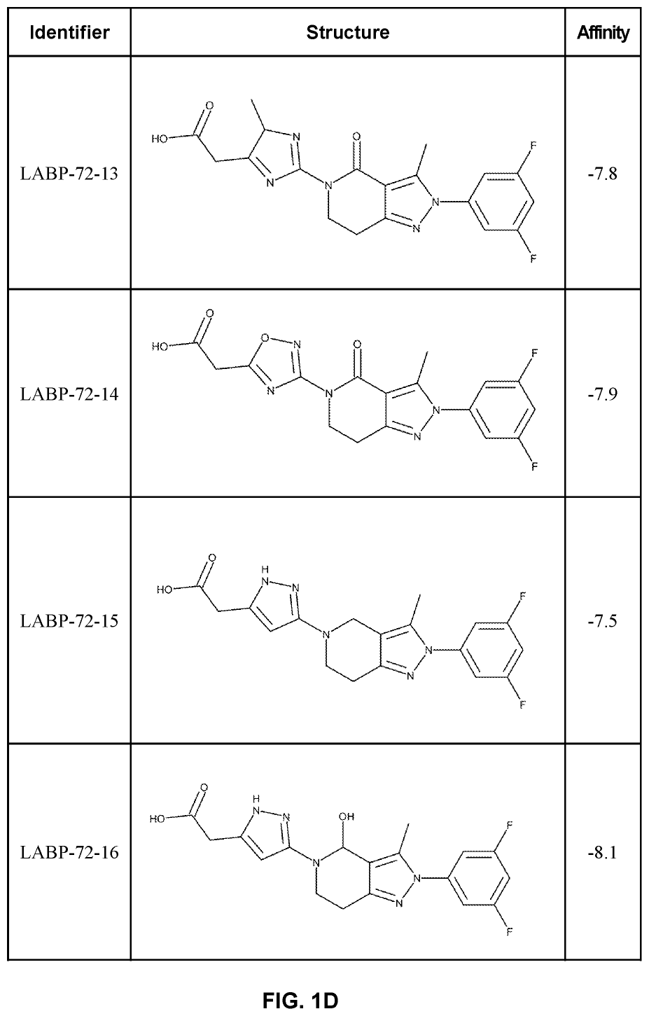

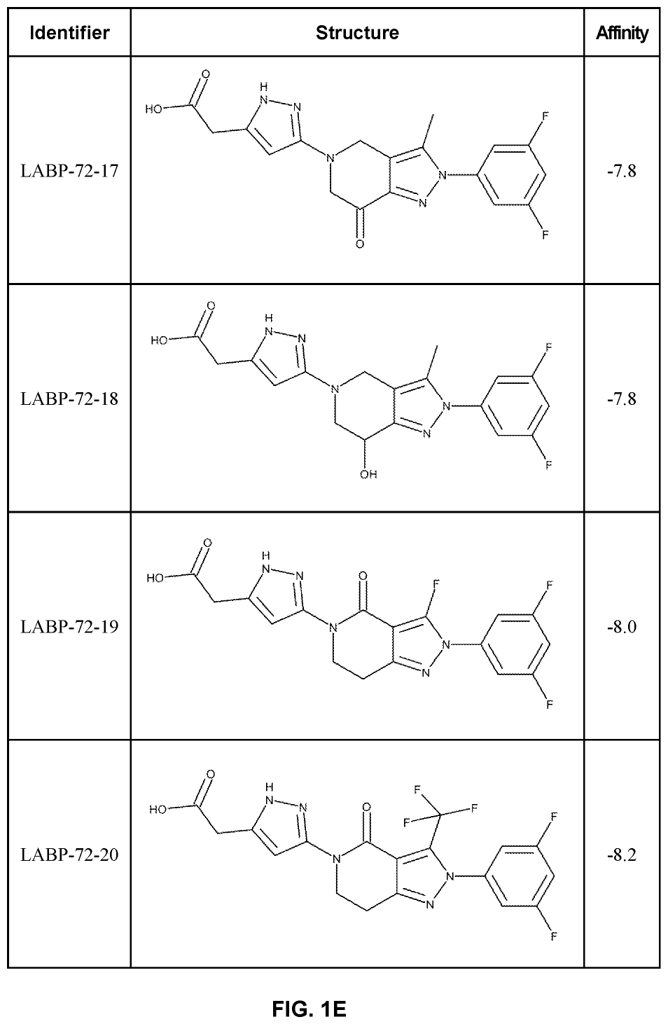

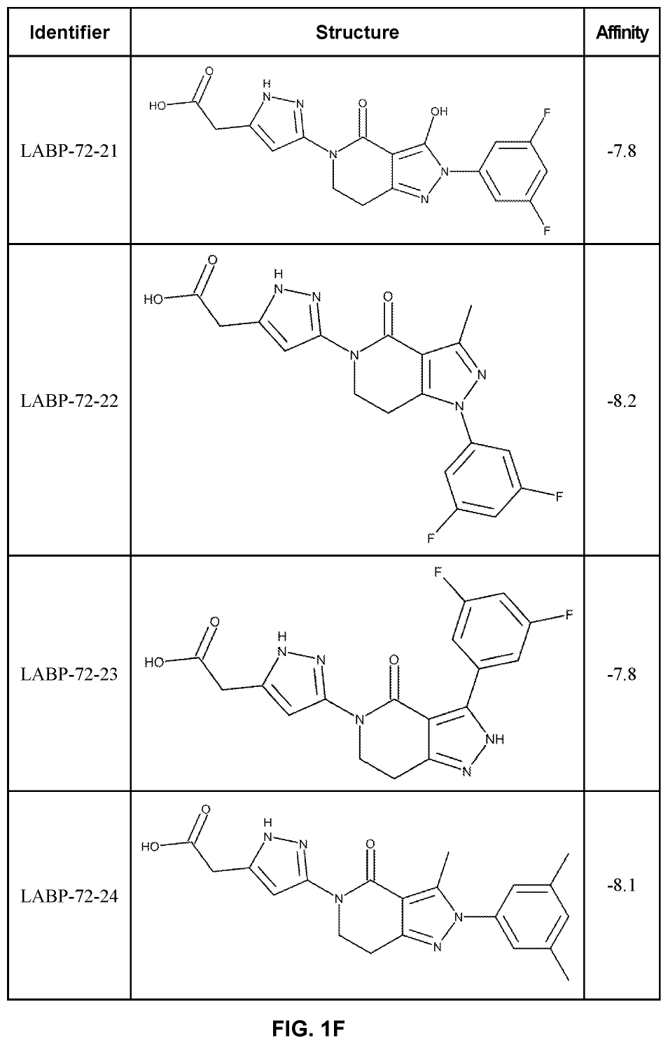

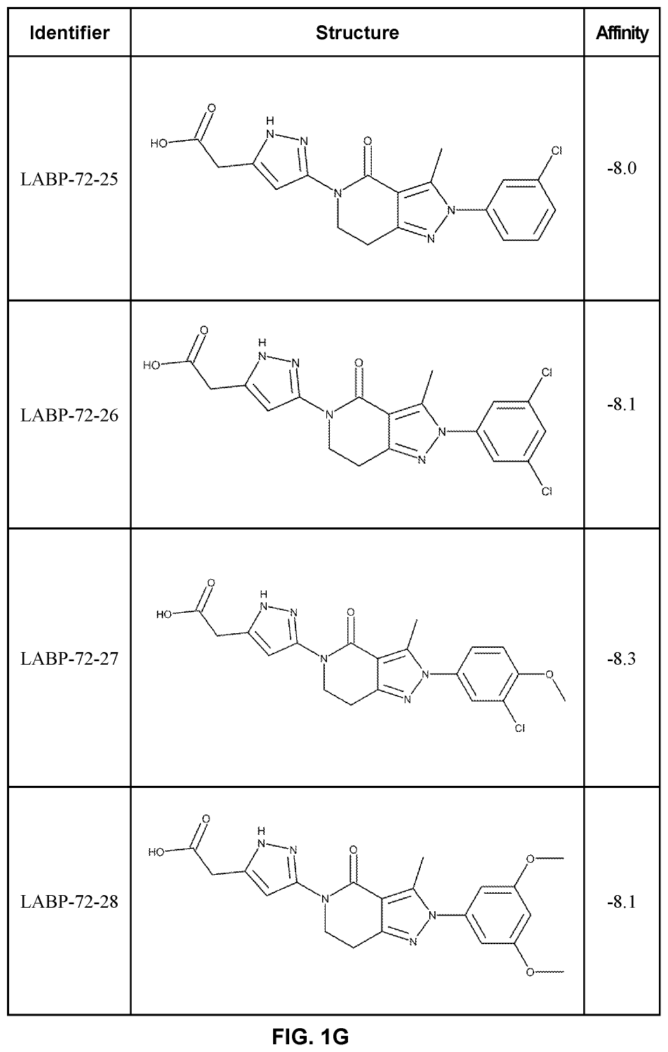

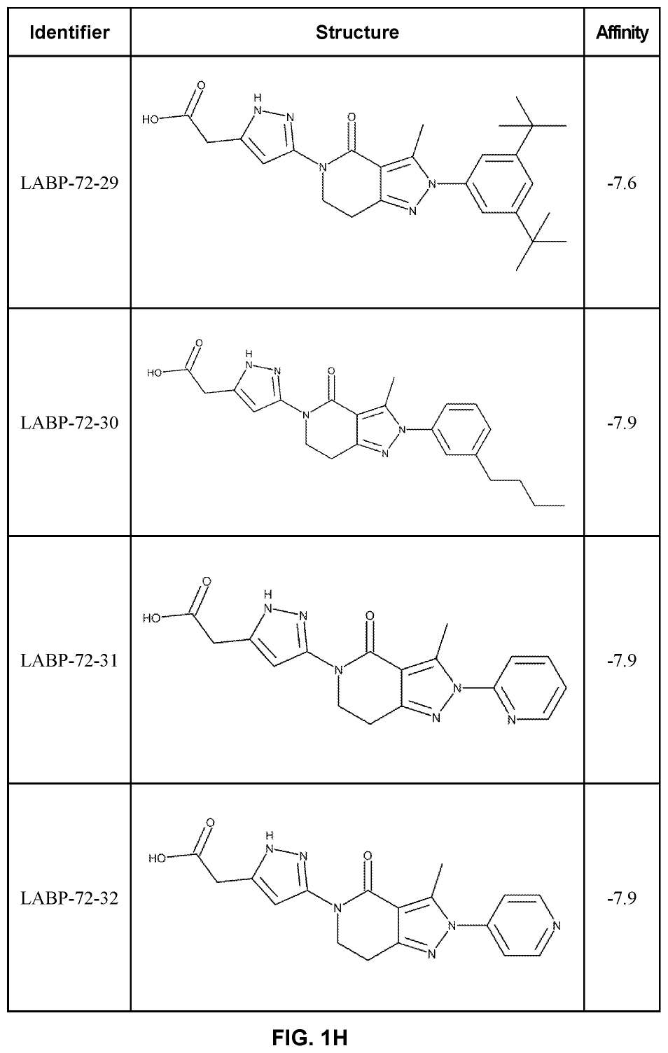

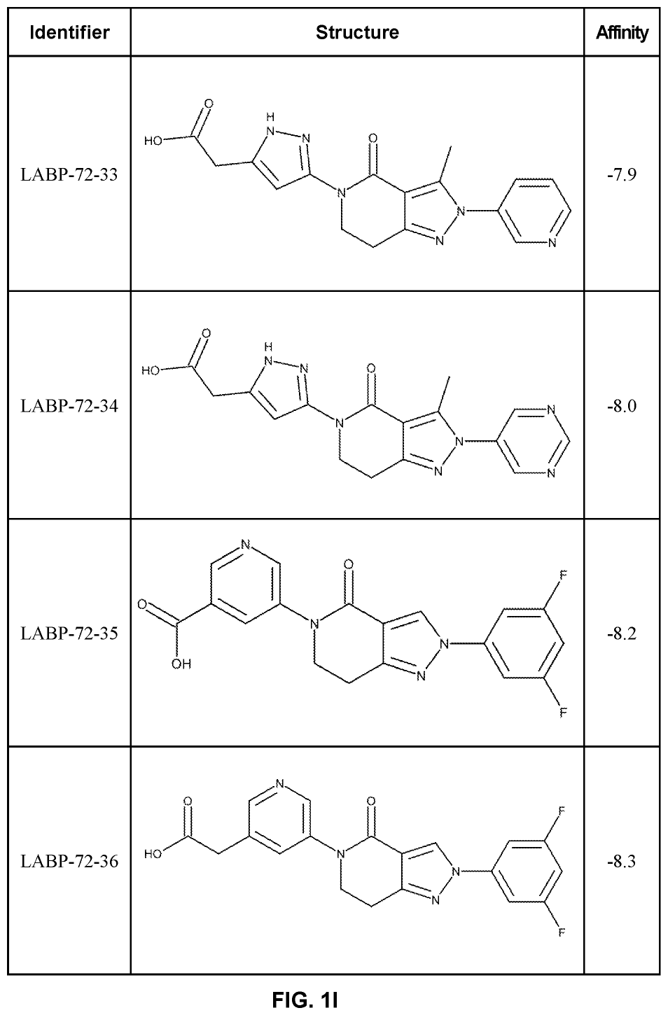

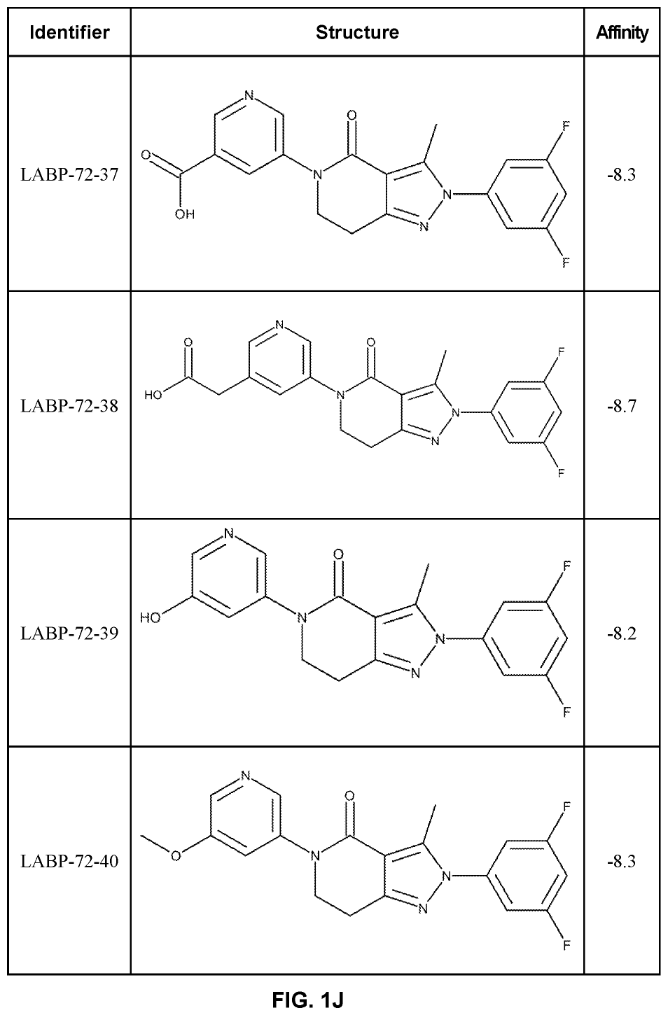

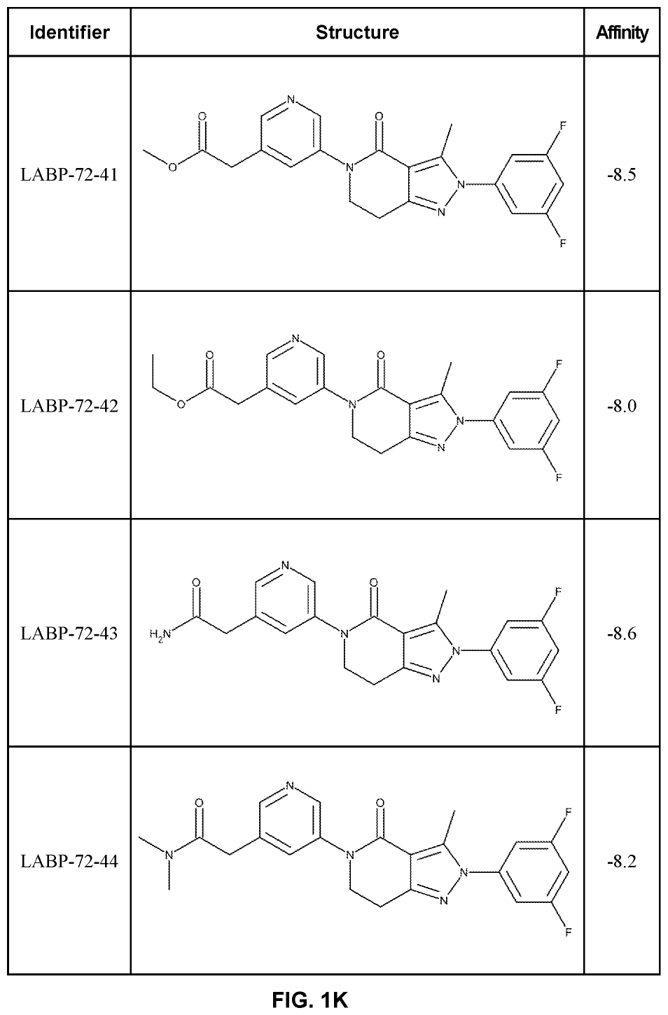

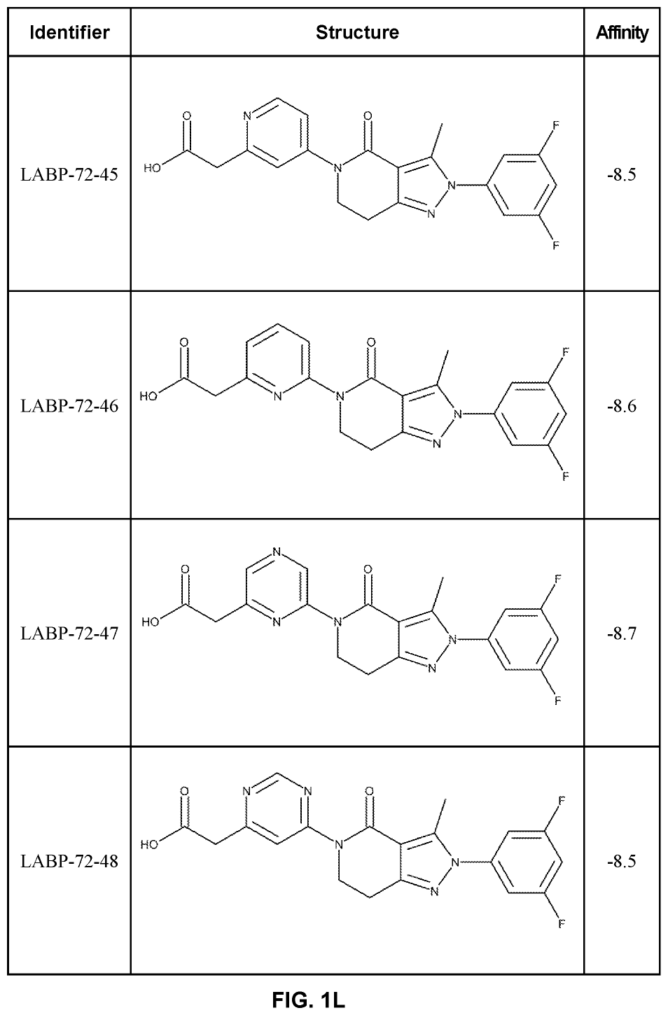

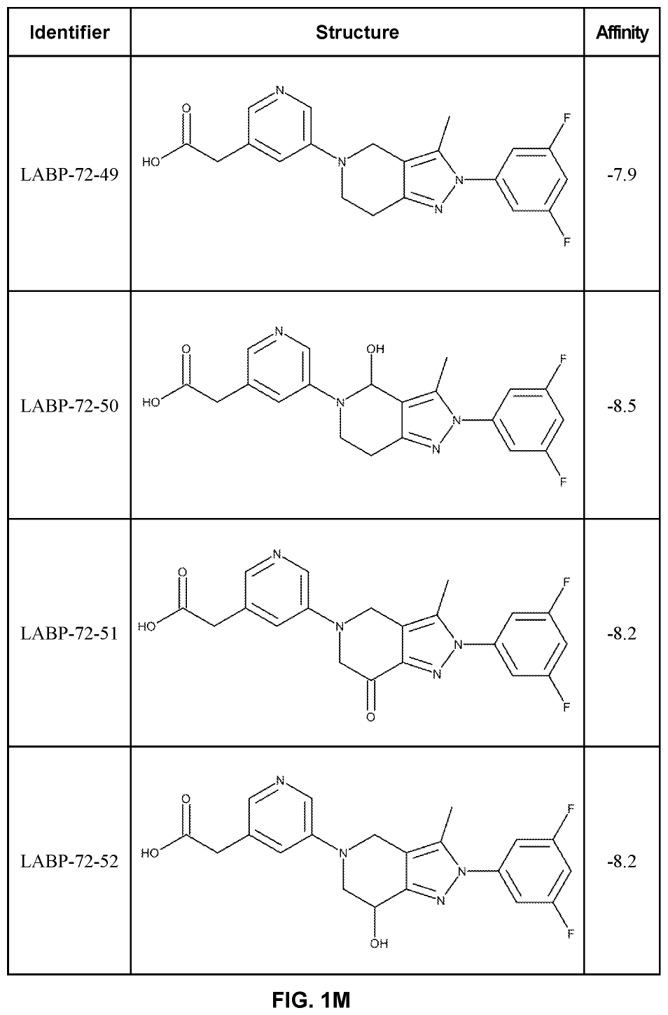

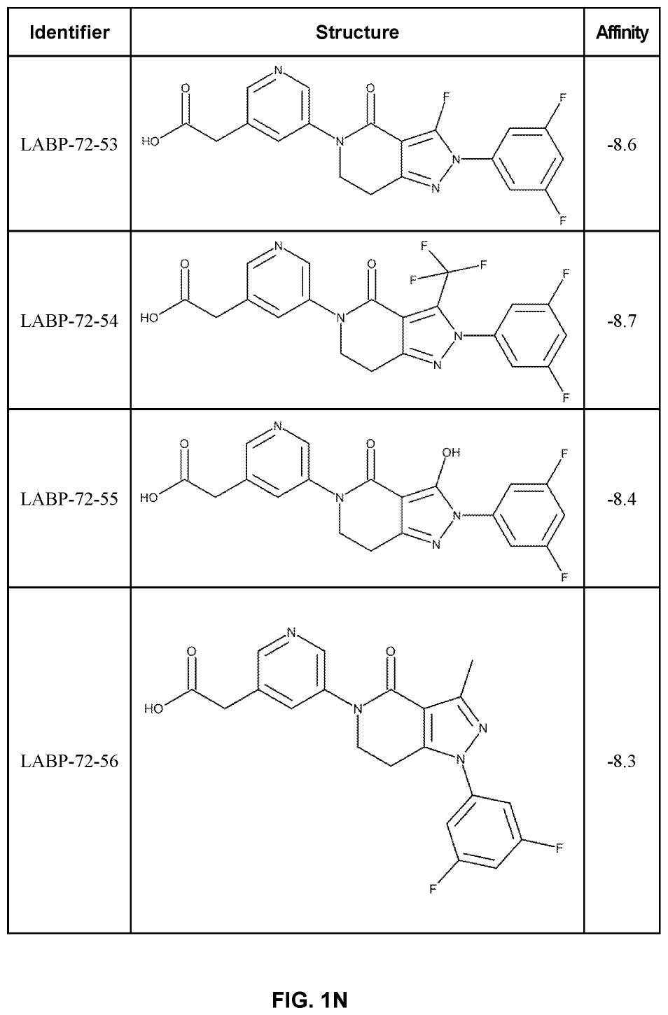

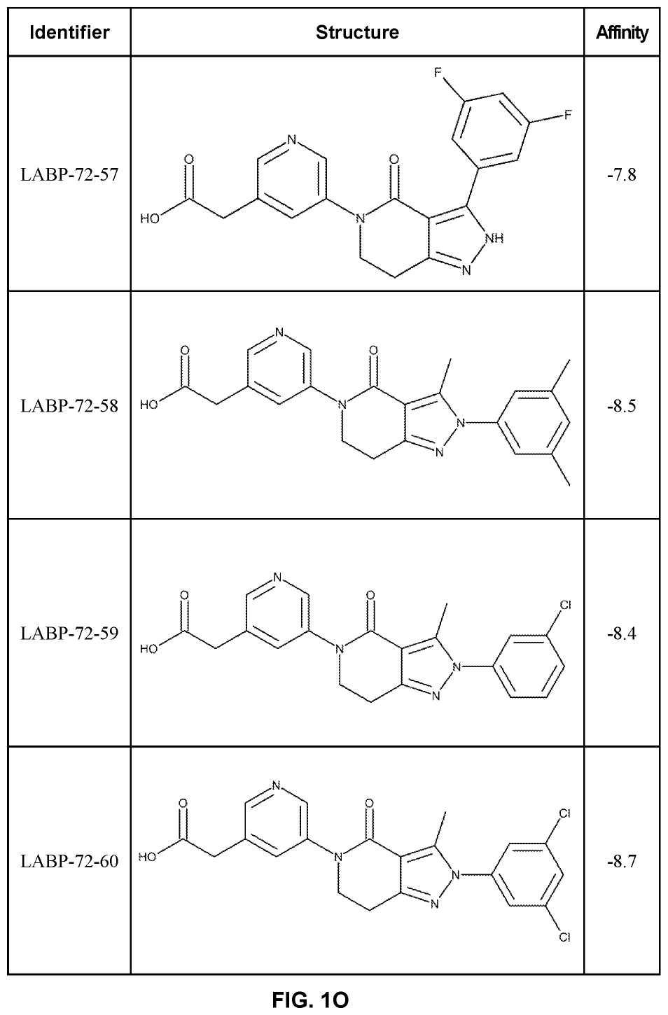

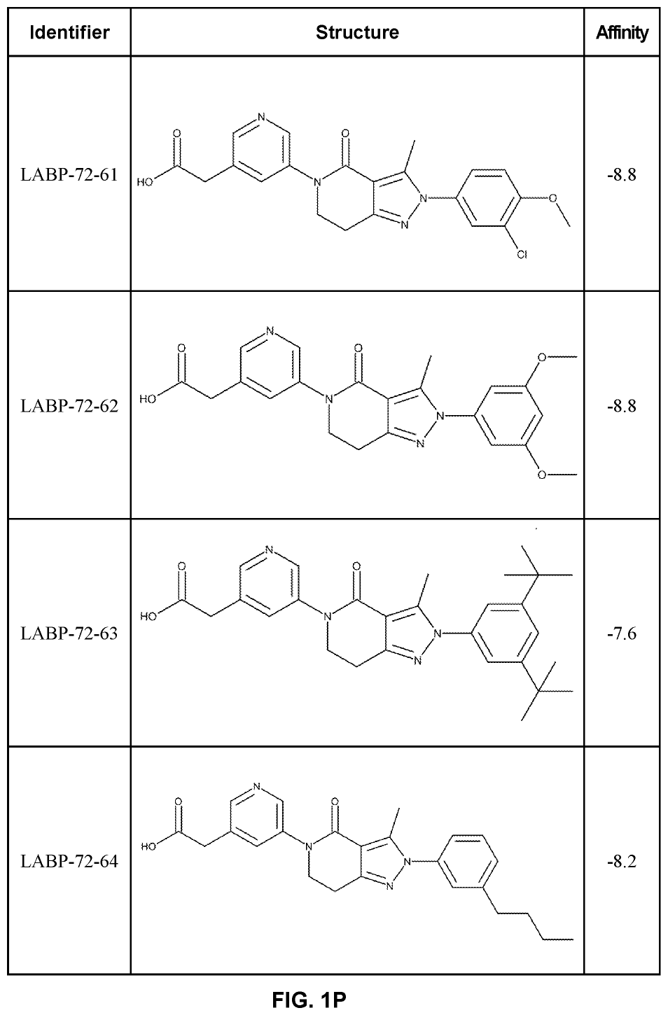

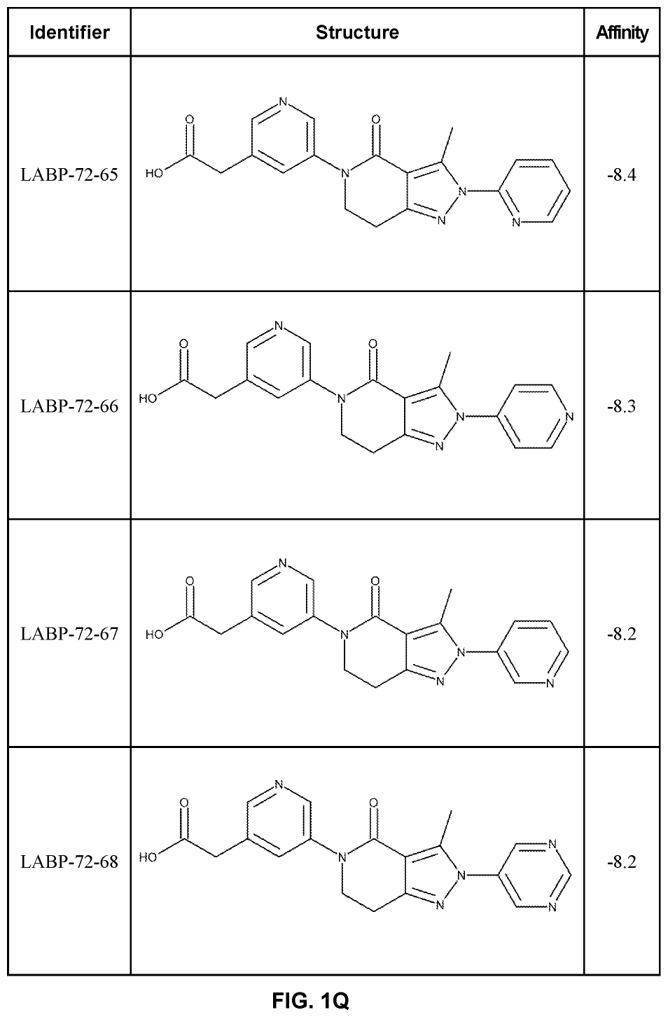

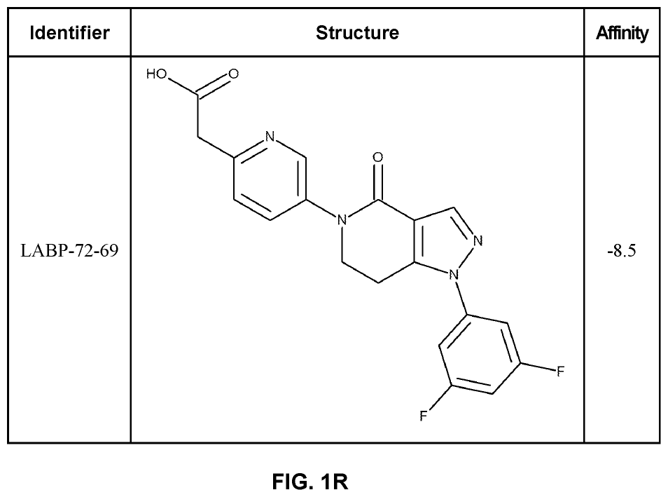

From the virtual screening and optimization of new chemical entities (NCEs), the highest affinity NLRX1-binding NCEs were largely comprised of compounds with a central 1,5,6,7-tetrahydropyrazolo[4,3-C]pyridin-4-one ring system. In general, binding affinities were observed to be increased in compounds that contained acetic acid moieties in the Y- group ring structure, a hydrophobic moiety added to the central group (i.e., the group formed by A¹, A², A³, A⁴, A⁵, A⁶, A⁷, A⁸, and A⁹ in Formula I), and a di-substituted Z-group ring structure. The binding affinities of selected family members are provided in FIGS. 1A-1R. The predicted binding affinities in the respective lowest energy binding configuration ranged from -7.5 kcal/mol to -8.8 kcal/mol. The highest binding compounds in this class of NCEs were observed to be LABP-72-61 and LABP-72-62 at −8.8 kcal/mol followed by LABP-72-38, LABP-72-47, LABP-72-54, and LABP-72-60 at −8.7 kcal/mol. Based on binding results and predicted physicochemical properties compounds were selected from this class for synthesis.

### Medicinal Chemistry

### Example 2. LABP-72-38

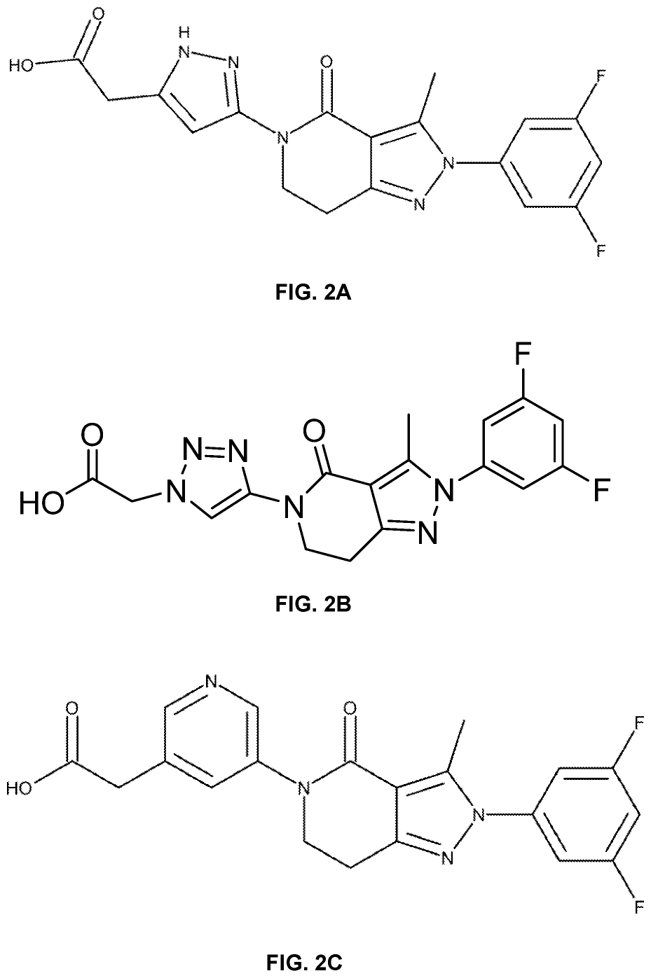

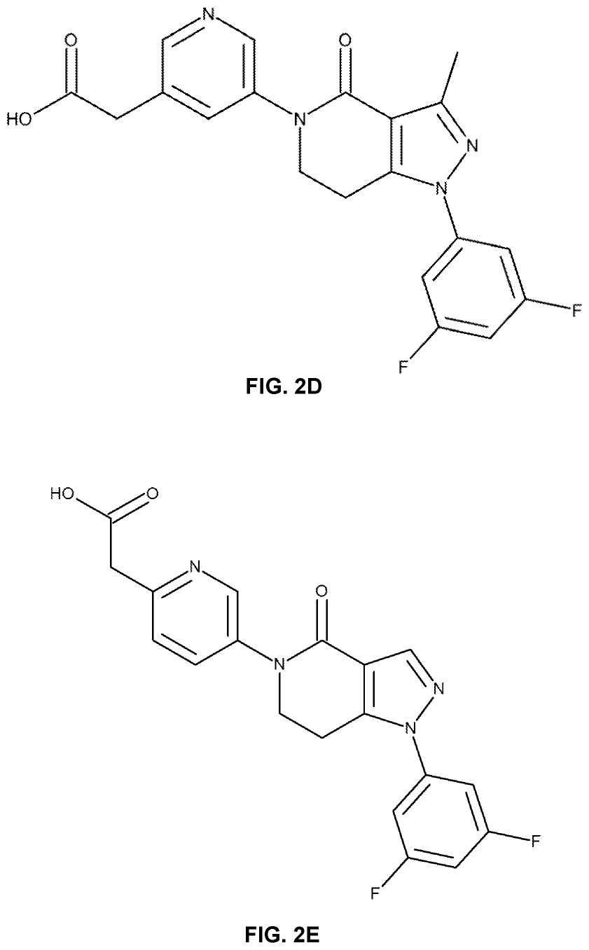

The synthesis of 2-(5-(2-(3,5-difluorophenyl)-4-oxo-2,4,6,7-tetrahydro-5H-pyrazolo[4,3-c]pyridin-5-yl)pyridin-3-yl)acetic acid (LABP-72-38, FIG. 2B) was a four step process as detailed below.

Piperidine-2,4-dione in ethanol was added to (3,5-difluorophenyl)hydrazine hydrochloride in acetic acid. The reaction mass was refluxed for 3 h and kept at room temperature for 24 h. After completion of reaction, solvent was evaporated under reduced pressure to obtain crude product. Crude product was diluted with water 10 mL and stirred for 10 min to precipitate solid. Solid was filtered and dried under vacuum to afford desired product as off-white solid.

N,N-Dimethylformamide dimethylacetal (DMF-DMA) was added to isolated product in dry DMF. The reaction mass was heated to 120° C. and stirred for 4 h. After completion of reaction, solvent was evaporated under reduced pressure to get crude product, which was azeotroped with toluene to obtain solid. Obtained solid was washed with hexane two times to obtain desired product.

Ethyl 2-(5-bromopyridin-3-yl)acetate and Cs₂CO₃ was added to a solution of product in 1,4-dioxane with purged solvent with nitrogen gas for 5 min. Pd₂(dba)₃ and Xanthphos were added and heated at 100° C. for 16 h. After completion of reaction, reaction mass was diluted with ethyl acetate and stirred for 10 min and filtered through celite, filtrate was evaporated to obtain crude product. Crude product was purified by column chromatography to afford desired product as pale yellow solid.

Lithium hydroxide was added to a solution of 2-(5-(2-(3,5-difluorophenyl)-4-oxo-2,4,6,7-tetrahydro-5H-pyrazolo[4,3-c]pyridin-5-yl)pyridin-3-yl)ethyl acetate in 1,4-dioxane and water and stirred at RT for 3 h. Solvent was evaporated to get crude residue. Crude residue was diluted with water and pH was adjusted to 2 using 2N HCl to precipitate solid. Precipitated solid was filtered and dried under vacuum to obtain crude product. Crude product was purified by reverse phase purification to afford 2-(5-(2-(3,5-difluorophenyl)-4-oxo-2,4,6,7-tetrahydro-5H-pyrazolo[4,3-c]pyridin-5-yl)pyridin-3-yl)acetic acid as off white solid (LABP-72-38).

### Example 3. LABP-72-69

The synthesis of 2-(5-(1-(3,5-difluorophenyl)-4-oxo-1,4,6,7-tetrahydro-5H-pyrazolo[4,3-c]pyridin-5-yl)pyridin-2-yl)acetic acid (LABP-72-69, FIG. 2D) was a four step process as detailed below.

DMF-DMA was added to a solution of piperidine-2,4-dione in dry DMF. The reaction mass was heated to 120° C. and stirred for 4 h. After completion of reaction, solvent was evaporated under reduced pressure to get crude, which was azeotroped with toluene to obtain dark brown solid. Solid was washed with hexane two times to obtain desired product as dark brown solid.

(3,5-Difluorophenyl)hydrazine hydrochloride was added to a solution of 3-((dimethylamino)methylene)piperidine-2,4-dione in ethanol. The reaction mass was heated to 120° C. for 16 h. After completion of reaction, solvent was evaporated under reduced pressure to obtain crude product. Crude product was diluted with water and stirred for 10 min to precipitate solid. Solid was filtered and dried under vacuum to afford desired product as off white solid.

Methyl 2-(5-bromopyridin-2-yl)acetate, xantphos ((9,9-Dimethyl-9H-xanthene-4,5-diyl)bis(diphenylphosphane)), and Cs₂CO₃ were added to a solution of filtered solid in 1,4- dioxane. Solvent was purged with nitrogen gas for 5 min. Pd₂(dba)₃ was added and heated at 100° C. for 16 h. After completion of reaction, the reaction mass was diluted with ethyl acetate and stirred for 10 min, filtered through celite and evaporated filtrate to obtain crude, which was purified by column chromatography to afford desired product as pale yellow solid.

Lithium hydroxide was added to a solution of 2-(5-(1-(3,5-difluorophenyl)-4-oxo-1,4,6,7-tetrahydro-5H-pyrazolo[4,3-c]pyridin-5-yl)pyridin-2-yl)methyl acetate in 1,4-dioxane and stirred at RT for 3 h. Reaction mixture was evaporated to obtain crude residue. Crude residue was diluted with water and acidified to pH 2 using 2N HCl, to precipitate solid, which was filtered and dried under vacuum to obtain crude product. Crude product was subjected to HPLC purification, pooled fractions were evaporated to afford 2-(5-(1-(3,5-difluorophenyl)-4-oxo-1,4,6,7-tetrahydro-5H-pyrazolo[4,3-c]pyridin-5-yl)pyridin-2-yl)acetic acid as off white solid (LABP-72-69).

### Example 4. LABP-72-56

The synthesis of 2-(5-(1-(3,5-difluorophenyl)-4-oxo-1,4,6,7-tetrahydro-5H-pyrazolo[4,3-c]pyridin-5-yl)pyridin-3-yl)acetic acid (LABP-72-56, FIG. 2C) was a four step process as detailed below.

DMF-DMA was added to a solution of piperidine-2,4-dione in dry DMF and heated to 120° C. and stirred for 4 h. After completion of reaction, solvent was evaporated under reduced pressure to get crude product. Crude product was azeotroped with toluene to obtain dark brown solid, which was washed with hexane two times to obtain desired product as dark brown solid.

(3,5-Difluorophenyl)hydrazine hydrochloride was added to a solution of 3-((dimethylamino) methylene)piperidine-2,4-dione in ethanol. The reaction mass was heated to 120° C. for 16 h. After completion of reaction, solvent was evaporated under reduced pressure to obtain crude product. Crude product was diluted with water and stirred for 10 min to precipitate solid. Solid was filtered and dried under vacuum to afford desired product as off white solid. Methyl 2-(5-bromopyridin-3-yl)acetate, xantphos, and Cs₂CO₃ were added to a solution of filtered solid in 1,4-dioxane. Solvent was purged with nitrogen gas for 5 min. Pd₂(dba)₃ was added and heated at 100° C. for 16 h. After completion of reaction, the reaction mass was diluted with ethyl acetate and stirred for 10 min, filtered through celite and evaporated filtrate to obtain crude, which was purified by column chromatography to afford desired product as pale yellow solid.

Lithium hydroxide was added to a solution of 2-(5-(1-(3,5-difluorophenyl)-4-oxo-1,4,6,7-tetrahydro-5H-pyrazolo[4,3-c]pyridin-5-yl)pyridin-3-yl)methyl acetate in 1,4-dioxane and stirred at RT for 3 h. Reaction mixture was evaporated to obtain crude residue. Crude residue was diluted with water and acidified to pH 2 using 2N HCl, to precipitate solid, which was filtered and dried under vacuum to obtain crude product. Crude product was subjected to HPLC purification, pooled fractions were evaporated to afford 2-(5-(1-(3,5-difluorophenyl)-4-oxo-1,4,6,7-tetrahydro-5H-pyrazolo[4,3-c]pyridin-5-yl)pyridin-3-yl)acetic acid as off white solid (LABP-72-56).

### Example 5. LABP-72-4

The synthesis of 2-(5-(2-(3,5-difluorophenyl)-4-oxo-2,4,6,7-tetrahydro-5H-pyrazolo[4,3-c]pyridin-5-yl)pyridin-3-yl)acetic acid (LABP-72-4, FIG. 2A) was a four step process as detailed below.

Piperidine-2,4-dione in cthanol was added to (3,5-difluorophenyl)hydrazine hydrochloride in acetic acid. The reaction mass was refluxed for 3 h and kept at room temperature for 24 h. After completion of reaction, solvent was evaporated under reduced pressure to obtain crude product. Crude product was diluted with water 10 mL and stirred for 10 min to precipitate solid. Solid was filtered and dried under vacuum to afford desired product as off-white solid.

DMF-DMA was added to isolated product in dry DMF. The reaction mass was heated to 120° C. and stirred for 4 h. After completion of reaction, solvent was evaporated under reduced pressure to get crude product, which was azcotroped with toluene to obtain solid. Obtained solid was washed with hexane two times to obtain desired product.

Methyl 2-(3-hydroxy-1H-pyrazol-5-yl)acetate was added to a solution of product in THF with DIAD and Ph₃P. After completion of reaction, reaction mass was diluted with ethyl acetate and stirred for 10 min and filtered through celite, filtrate was evaporated to obtain crude product. Crude product was purified by column chromatography to afford desired product as pale yellow solid.

Lithium hydroxide was added to a solution of 2-(5-(2-(3,5-difluorophenyl)-4-oxo-2,4,6,7-tetrahydro-5H-pyrazolo[4,3-c]1H-pyrazol[2,3-yl]-5-yl)methyl acetate in methanol and water and stirred at RT for 3 h. Solvent was evaporated and solid was filtered and dried under vacuum to obtain crude product. Crude product was purified by reverse phase purification to afford 2-(5-(2-(3,5-difluorophenyl)-4-oxo-2,4,6,7-tetrahydro-5H-pyrazolo[4,3-c] 1H-pyrazol[2,3-yl]-5-yl)acetic acid as off white solid (LABP-72-4).

### Experimental Studies

### Example 6. Immunological Screening In Vitro in CD4+ T Cells

Introduction

CD4+T cells are central to the pathogenesis of many autoimmune diseases and the amplification of inflammatory responses that can contribute to organ damage. As such, the trafficking and differentiation of these cells is an effective option for the amelioration of symptoms and prevention of flares in autoimmune disease. With the loss of NLRX1, CD4+ T cells produced greater amounts of IFNγ and TNFα and have a higher likelihood of differentiating into inflammatory subsets, such as Th17 and Th1.

### Methods

Cell culture. Spleens were excised from C57BL/6 mice. Spleens were crushed between the frosted ends of microscope slides and filtered to provide a cellular suspension. Red blood cells were lysed through hypotonic lysis. Remaining cells were washed and filtered. CD4+ T cells were enriched within the suspension using magnetic sorting based negative selection. Cells were collected and plated within 96 well plates coated with anti-CD3/CD28 and cultured in the presence of LABP-72-4, LABP-72-38, LABP-72-56, LABP-72-69 at 0 or 100 nanomolar for 24 h. During the last 6 h of culture, cells were stimulated with phorbol 12-myristate-13-acetate (PMA) and ionomycin.

Immunological analysis. Cells were collected from 96 well plates and stained with a cocktail of antibodies for immunophenotyping by flow cytometry. Culture supernatant was collected and assayed for cytokine concentrations by cytometric bead array. Data was captured on a BD FACS Celesta and analyzed using FACSDiva.

### Results

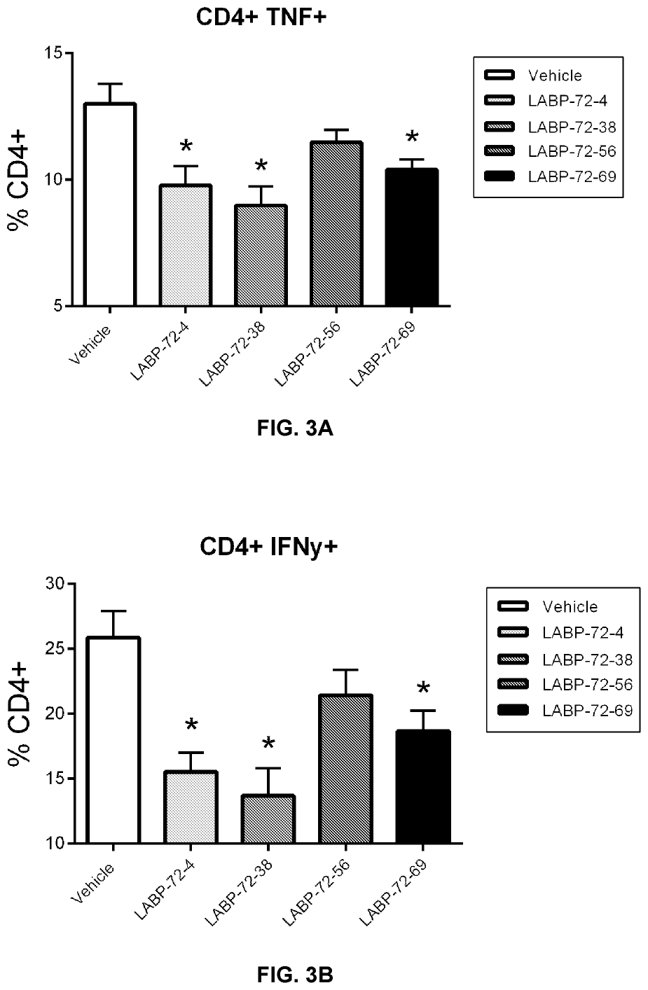

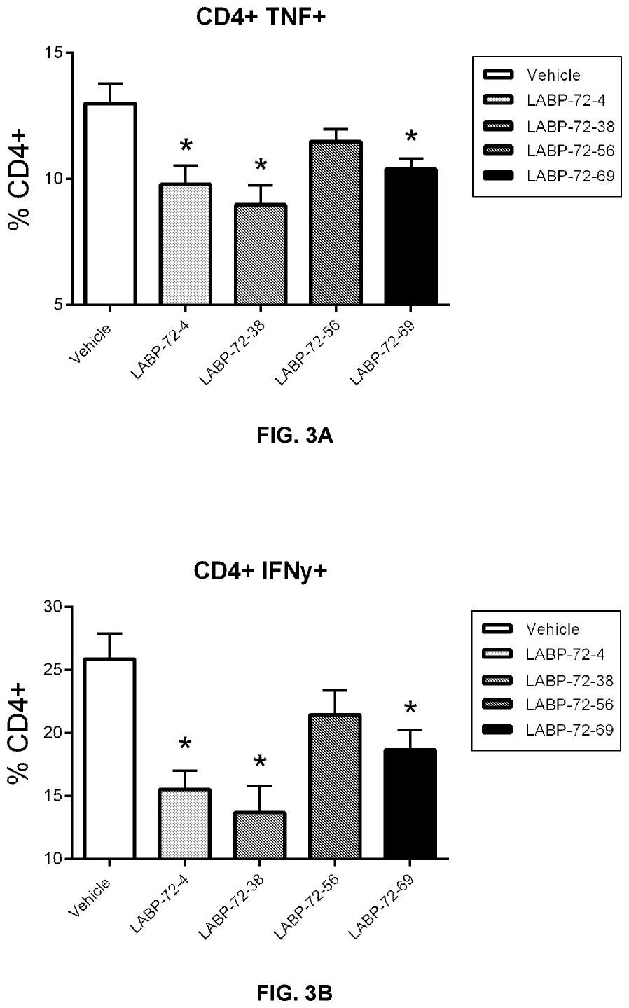

The four tested NLRX1 ligands all decreased production of TNFα (FIG. 3A) and IFNγ (FIG. 3B) in CD4+ T cell culture. LABP-72-4, LABP-72-38, and LABP-72-69 were observed to have the largest magnitude of response, significantly reducing both metrics relative to vehicle control.

### Example 7. Use of LABP-72-38 in Models of Asthma

Asthma is a common disease affecting nearly 10% of the population with high proportions of patients unresponsive to current medications. In particular, non-type 2 asthma has a lower responsiveness to current treatments. Defects in airway epithelial cells, increased neutrophil recruitment and underlying pulmonary fibrosis create a more complex pathogenesis in many refractory patients relative to allergic asthma. Previously, the loss of NLRX1 has been identified to disrupt metabolism and cause cell death in airway epithelial cells and increase neutrophil recruitment in a variety of inflammatory conditions.

### Methods

OVA-induced model. BALB/c mice were immunized with 10 μg of ovalbumin (OVA) in aluminum hydroxide gel by intraperitoneal injection on day 0 and 7 of the experiment. Mice were then exposed to OVA (8% w/v) by aerosolization for 25 minutes daily between days 14 and 17. Treatment with LABP-72-38 (50 mg/kg) or vehicle control occurred daily between days 14 and 17 by oral gavage. An unchallenged negative control group was included for comparison. Dosage was calculated based off mean body weights.

HDM-induced model. BALB/c mice were sensitized to house dust mite with 25 μg administered daily by intranasal instillation for five days. Challenge with house dust mite (25 μg/d) occurred for 5 consecutive days for the following three weeks. Treatment with LABP-72-38 (20 mg/kg) or vehicle control occurred daily during this three-week period. An unchallenged negative control group was included for comparison.

Immunological analysis. Lungs were collected on day 18 (OVA) or day 27 (HDM). Lung tissue was minced and digested in RPMI supplemented with FBS, HEPES, and calcium chloride containing 300 U/mL collagenase and 50 U/mL DNase for 45 minutes at 37° C. After filtration, red blood cells were lysed. Cells were labeled with mixtures of extracellular (CD45, CD3, CD4, CD8, MHCII, CD11b, CD11c, SiglecF, Ly6C, Ly6G) antibodies in a sequential live staining in 96-well plates in preparation for flow cytometry. Data was captured on a BD FACS Celesta and analyzed using FACSDiva.

### Results

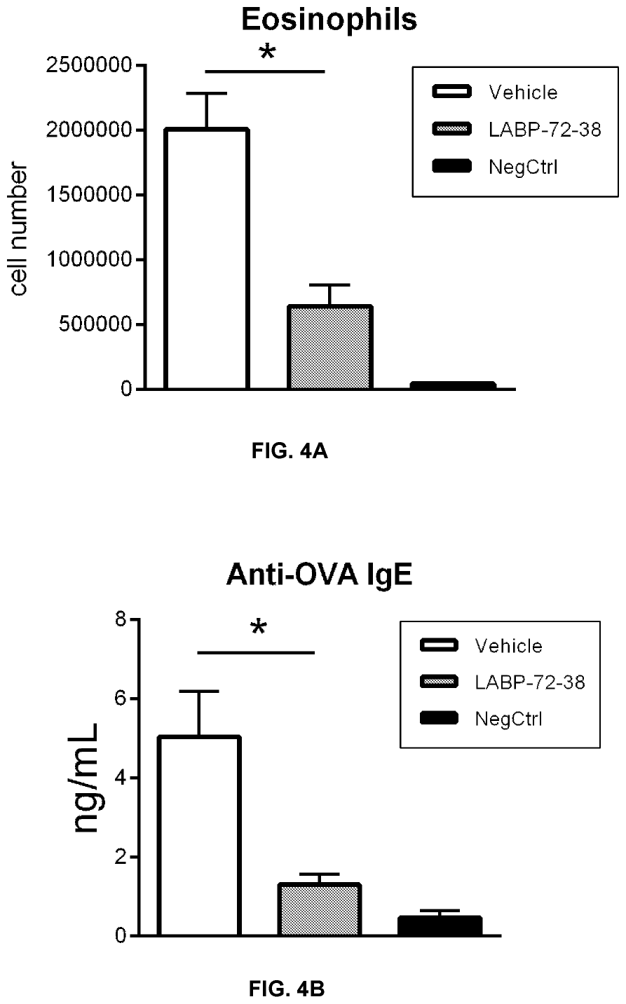

Oral LABP-72-38 reduced the percentage of cosinophils (FIG. 4A) within the lung on day 18 as well as reducing the concentrations of anti-OVA IgE antibodies in the serum (FIG. 4B) on day 18 relative to vehicle control, suggesting the potential for NLRX1 ligands to decrease pulmonary inflammation.

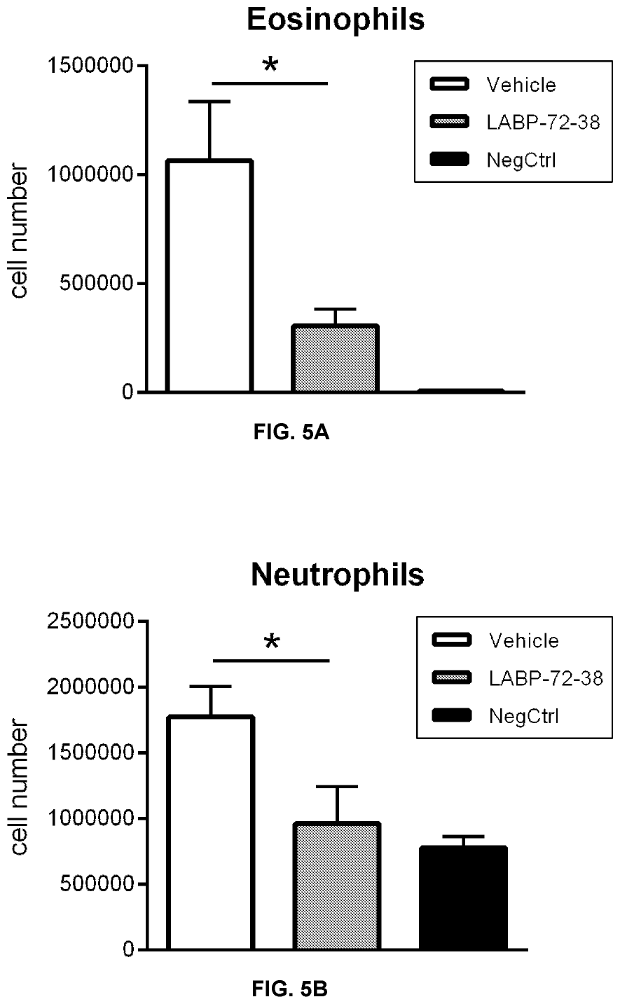

Intranasal LABP-72-38 reduced the percentage of cosinophils (FIG. 5A) and neutrophils (FIG. 5B) within the lung on day 18 relative to vehicle control, further suggesting the potential for NLRX1 ligands to decrease pulmonary inflammation.

### Example 8. Use of LABP-72-38 in a Bleomycin-Induced Model of Pulmonary Fibrosis

### Introduction

Idiopathic pulmonary fibrosis is a progressive disease in which the connective tissue and alveoli of the lung become scarred and thickened. Over time, this causes the lungs to function less efficiently, leading to shortness of breath, oxygen deficiency and eventually death. Idiopathic pulmonary fibrosis is estimated to affect around 100,000 individuals in the United States with 30,000-40,000 new cases annually. The exact cause of idiopathic pulmonary fibrosis is unknown; however, the progression of the disease is thought to be connected to chronic inflammation. Much of this inflammation is derived from myeloid cells that contribute to modulating levels of cytokines, growth factors and other molecules tied to extracellular matrix organization. In the context of other inflammatory diseases, NLRX1 has been shown to be important in the regulation of fibrosis and is strongly tied to the regulation of pulmonary inflammation.

### Methods

Bleomycin model. C57BL/6 mice were anesthetized by isoflurane and intratracheally administered 1 mg/kg bleomycin at day 0. LABP-72-38 was prepared within a 0.5% methylcellulose (12-15 cP) solution. Dosage used was 50 mg/kg delivered once daily, by oral gavage beginning 7 days after bleomycin challenge. Lungs were collected 2 weeks after bleomycin challenge.

Flow Cytometry. Lungs were collected into RPMI/FBS buffer containing collagenase (300U/mL) and DNase (50U/mL) for digestion. Tissues were digested for 45 minutes under stirring at 37° C. Resultant cellular suspensions were filtered through 100 μm strainers, and centrifuged (300× g, 8 min). Red blood cells were lysed from the cellular suspensions. Cells were filtered and washed in fresh RPMI. Cells were labeled with mixtures of extracellular (CD45, CD3, CD4, CD8, CD19, NK1.1, F4/80, CD11b, Gr1) and intracellular (IL21) antibodies in a sequential live staining in 96-well plates. Data was acquired using a FACS Celesta flow cytometer with FACSDiva software.

### Results

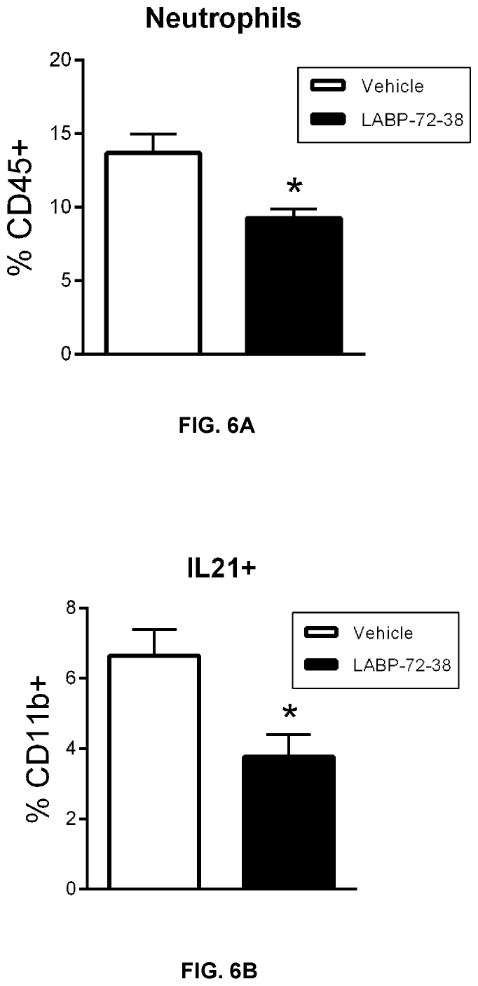

Oral LABP-72-38 treatment decreased the proportions of neutrophils (FIG. 6A) and IL21+ cells (FIG. 6B) in the lungs after bleomycin challenge, relative to vehicle treated controls. Both cell types are associated with worsened pulmonary function and increased fibrosis.

### Example 9. Use of LABP-72-38 in a High Fat Diet-Induced Model of Obesity

Pre-diabetes and metabolic syndrome are two serious health conditions that afflict about 40.1% of adults in the U.S. and dramatically increase the risk for type 2 diabetes (T2D), cardiovascular disease and stroke. Nearly half of U.S. adults have pre-diabetes and T2D, a disease that has reached pandemic proportions with over 400 million people afflicted worldwide. In the U.S., one of every nine dollars spent on healthcare is put toward T2D management and care. Although poor diet and obesity are significant risk factors for T2D, most people are not adherent to heathy lifestyle. Recent evidence suggests that chronic low-grade inflammation persists in overweight and obese individuals. This inflammation may result from biasing of immune cells into inflammatory states due to the metabolic microenvironment to which they are exposed. NLRX1 is a receptor that we predict will help to reverse this bias.

### Methods

Mouse model. C57BL6 mice were challenged with 16 weeks of high fat feeding by ad libitum exposure to rodent diet chow containing 60% kcal from fat. After 12 weeks of challenge, mice were treated daily with LABP-72-38 at 10 mg/kg for 4 weeks. Treatment was delivered by oral gavage. Mice were weighed weekly. Blood glucose levels were measured by glucometer on blood collected from the tail vein. After 16 weeks of diet, livers were excised and weighed. Weights were normalized to the total body weight.

### Results

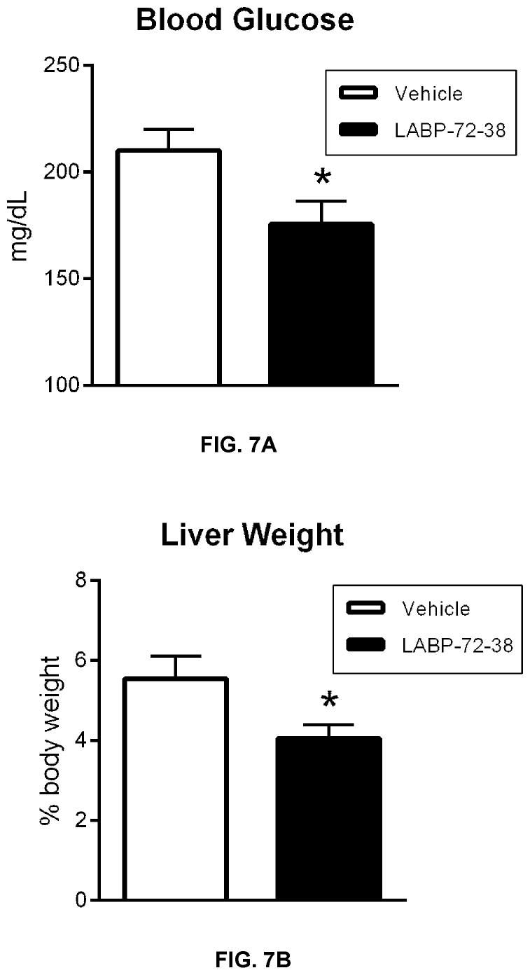

Oral LABP-72-38 reduced the blood glucose level of mice consuming a high-fat diet (FIG. 7A). LABP-72-38 also reduced body weight normalized liver weight when compared to vehicle treated controls (FIG. 7B).

### REFERENCES

Abreu, M. T., Toll-like receptor signalling in the intestinal epithelium: how bacterial recognition shapes intestinal function. Nat Rev Immunol, 2010. 10(2): p. 131-144.
Allen, I. C., C. B. Moore, M. Schneider, Y. Lei, B. K. Davis, M. A. Scull, D. Gris, K. E. Roney, A. G. Zimmermann, J. B. Bowzard, P. Ranjan, K. M. Monroe, R. J. Pickles, S. Sambhara, and J. P. Ting, NLRX1 protein attenuates inflammatory responses to infection by interfering with the RIG-I-MAVS and TRAF6-NF-kappaB signaling pathways. Immunity, 2011. 34(6): p. 854-865.
Arnoult, D., F. Soares, I. Tattoli, C. Castanier, D. J. Philpott, and S. E. Girardin, An N-terminal addressing sequence targets NLRX1 to the mitochondrial matrix. J Cell Sci, 2009. 122(Pt 17): p. 3161-3168.
Costford, S. R., I. Tattoli, F. T. Duan, A. Volchuk, A. Klip, D. J. Philpott, M. Woo, and S. E. Girardin, Male Mice Lacking NLRX1 Are Partially Protected From High-Fat Dict-Induced Hyperglycemia. J Endocr Soc, 2018. 2(4): p. 336-347.
Coutermarsh-Ott, S., A. Simmons, V. Capria, T. LeRoith, J. E. Wilson, B. Heid, C. W. Philipson, Q. Qin, R. Hontecillas-Magarzo, J. Bassaganya-Riera, J. P. Ting, N. Dervisis, and I. C. Allen, NLRX1 suppresses tumorigenesis and attenuates histiocytic sarcoma through the negative regulation of NF-kappaB signaling. Oncotarget, 2016. 7(22): p. 33096-33110.
Davis, B. K., C. Philipson, R. Hontecillas, K. Eden, J. Bassaganya-Riera, and I. C. Allen, Emerging significance of NLRs in inflammatory bowel disease. Inflamm Bowel Dis, 2014. 20(12): p. 2412-2432.
Eitas, T. K., W. C. Chou, H. Wen, D. Gris, G. R. Robbins, J. Brickey, Y. Oyama, and J.P. Ting, The nucleotide-binding leucine-rich repeat (NLR) family member NLRX1 mediates protection against experimental autoimmune encephalomyelitis and represses macrophage/microglia-induced inflammation. J Biol Chem, 2014. 289(7): p. 4173-4179.
Feng, H., E. M. Lenarcic, D. Yamane, E. Wauthier, J. Mo, H. Guo, D. R. McGivern, O. Gonzalez-Lopez, I. Misumi, L. M. Reid, J. K. Whitmire, J. P. Ting, J. A. Duncan, N. J. Moorman, and S. M. Lemon, NLRX1 promotes immediate IRF1-directed antiviral responses by limiting dsRNA-activated translational inhibition mediated by PKR. Nat Immunol, 2017. 18(12): p. 1299-1309.
Guo, H., R. Konig, M. Deng, M. Riess, J. Mo, L. Zhang, A. Petrucelli, S. M. Yoh, B. Barefoot, M. Samo, G. D. Sempowski, A. Zhang, A. M. Colberg-Poley, H. Feng, S. M. Lemon, Y. Liu, Y. Zhang, H. Wen, Z. Zhang, B. Damania, L.C. Tsao, Q. Wang, L. Su, J. A. Duncan, S. K. Chanda, and J. P. Ting, NLRX1 Sequesters STING to Negatively Regulate the Interferon Response, Thereby Facilitating the Replication of HIV-1 and DNA Viruses. Cell Host Microbe, 2016. 19(4): p. 515-528.
Hong, M., S. I. Yoon, and I. A. Wilson, Structure and functional characterization of the RNA-binding element of the NLRX1 innate immune modulator. Immunity, 2012. 36(3): p. 337-347.
Jaworska, J., F. Coulombe, J. Downey, F. Tzelepis, K. Shalaby, I. Tattoli, J. Berube, S. Rousseau, J. G. Martin, S. E. Girardin, J. A. McCullers, and M. Divangahi, NLRX1 prevents mitochondrial induced apoptosis and enhances macrophage antiviral immunity by interacting with influenza virus PB1-F2 protein. Proc Natl Acad Sci U S A, 2014. 111(20): p. E2110-2119.
Kale, S. D., T. Ayubi, D. Chung, N. Tubau-Juni, A. Leber, H. X. Dang, S. Karyala, R. Hontecillas, C .B. Lawrence, R. A. Cramer, and J. Bassaganya-Riera, Modulation of Immune Signaling and Metabolism Highlights Host and Fungal Transcriptional Responses in Mouse Models of Invasive Pulmonary Aspergillosis. Sci Rep, 2017. 7(1): p. 17096.
Kang, M. J., C. M. Yoon, B. H. Kim, C. M. Lee, Y. Zhou, M. Sauler, R. Homer, A. Dhamija, D. Boffa, A. P. West, G. S. Shadel, J. P. Ting, J. R. Tedrow, N. Kaminski, W. J. Kim, C. G. Lee, Y. M. Oh, and J. A. Elias, Suppression of NLRX1 in chronic obstructive pulmonary disease. J Clin Invest, 2015. 125(6): p. 2458-2462.
Kim, J. H., M. E. Park, C. Nikapitiya, T. H. Kim, M. B. Uddin, H. C. Lee, E. Kim, J. Y. Ma, J. U. Jung, C. J. Kim, and J. S. Lee, FAS-associated factor-1 positively regulates type I interferon response to RNA virus infection by targeting NLRX1. PLOS Pathog, 2017. 13(5): p. e1006398.
Koblansky, A. A., A. D. Truax, R. Liu, S. A. Montgomery, S. Ding, J. E. Wilson, W. J. Brickey, M. Muhlbauer, R. M. McFadden, P. Hu, Z. Li, C. Jobin, P. K. Lund, and J. P. Ting, The Innate Immune Receptor NLRX1 Functions as a Tumor Suppressor by Reducing Colon Tumorigenesis and Key Tumor-Promoting Signals. Cell Rep, 2016. 14(11): p. 2562-2575.
Kors, L., E. Rampanelli, G. Stokman, L. M. Butter, N. M. Held, N. Claessen, P. W. B. Larsen, J. Verheij, C. J. Zuurbier, G. Liebisch, G. Schmitz, S. E. Girardin, S. Florquin, R. H. Houtkooper, and J. C. Leemans, Deletion of NLRX1 increases fatty acid metabolism and prevents diet-induced hepatic steatosis and metabolic syndrome. Biochim Biophys Acta, 2018. 1864(5 Pt A): p. 1883-1895.
Leber, A., R. Hontecillas, N. Tubau-Juni, V. Zoccoli-Rodriguez, M. Hulver, R. McMillan, K. Eden, I. C. Allen, and J. Bassaganya-Riera, NLRX1 Regulates Effector and Metabolic Functions of CD4+ T Cells. J Immunol, 2017.
Leber, A., R. Hontecillas, N. Tubau-Juni, V. Zoccoli-Rodriguez, V. Abedi, and J. Bassaganya-Riera, NLRX1 Modulates Immunometabolic Mechanisms Controlling the Host-Gut Microbiota Interactions during Inflammatory Bowel Disease. Front Immunol, 2018. 9: p. 363.
Lei, Y., B. A. Kansy, J. Li, L. Cong, Y. Liu, S. Trivedi, H. Wen, J. P. Ting, H. Ouyang, and R. L. Ferris, EGFR-targeted mAb therapy modulates autophagy in head and neck squamous cell carcinoma through NLRX1-TUFM protein complex. Oncogene, 2016. 35(36): p. 4698-4707.
Li, H., S. Zhang, F. Li, and L. Qin, NLRX1 attenuates apoptosis and inflammatory responses in myocardial ischemia by inhibiting MAVS-dependent NLRP3 inflammasome activation. Mol Immunol, 2016. 76: p. 90-97.
Lu, P., R. Hontecillas, V. Abedi, S. Kale, A. Leber, C. Heltzel, M. Langowski, V. Godfrey, C. Philipson, N. Tubau-Juni, A. Carbo, S. Girardin, A. Uren, and J. Bassaganya-Riera, Modeling-Enabled Characterization of Novel NLRX1 Ligands. PLOS One, 2015. 10(12): p. e0145420.
Ma, Z., S. E. Hopcraft, F. Yang, A. Petrucelli, H. Guo, J. P. Ting, D. P. Dittmer, and B. Damania, NLRX1 negatively modulates type I IFN to facilitate KSHV reactivation from latency. PLOS Pathog, 2017. 13(5): p. e 1006350.
Moore, C. B., D. T. Bergstralh, J. A. Duncan, Y. Lei, T. E. Morrison, A. G. Zimmermann, M. A. Accavitti-Loper, V. J. Madden, L. Sun, Z. Ye, J. D. Lich, M. T. Heise, Z. Chen, and J. P. Ting, NLRX1 is a regulator of mitochondrial antiviral immunity. Nature, 2008. 451(7178): p. 573-577.
Philipson, C. W., J. Bassaganya-Riera, M. Viladomiu, B. Kronsteiner, V. Abedi, S. Hoops, P. Michalak, L. Kang, S. E. Girardin, and R. Hontecillas, Modeling the Regulatory Mechanisms by Which NLRX1 Modulates Innate Immune Responses to Helicobacter pylori Infection. PLOS One, 2015. 10(9): p. e0137839.
Singh, K., A. Poteryakhina, A. Zheltukhin, K. Bhatelia, P. Prajapati, L. Sripada, D. Tomar, R. Singh, A. K. Singh, P. M. Chumakov, and R. Singh, NLRX1 acts as tumor suppressor by regulating TNF-alpha induced apoptosis and metabolism in cancer cells. Biochim Biophys Acta, 2015. 1853(5): p. 1073-1086.
Soares, F., I. Tattoli, M. A. Rahman, S. J. Robertson, A. Belcheva, D. Liu, C. Streutker, S. Winer, D. A. Winer, A. Martin, D. J. Philpott, D. Arnoult, and S. E. Girardin, The mitochondrial protein NLRX1 controls the balance between extrinsic and intrinsic apoptosis. J Biol Chem, 2014. 289(28): p. 19317-19330.
Tattoli, I., S. A. Killackey, E. G. Foerster, R. Molinaro, C. Maisonneuve, M. A. Rahman, S. Winer, D. A. Winer, C. J. Streutker, D. J. Philpott, and S. E. Girardin, NLRX1 Acts as an Epithelial-Intrinsic Tumor Suppressor through the Modulation of TNF-Mediated Proliferation. Cell Rep, 2016. 14(11): p. 2576-2586.
Theus, M. H., T. Brickler, A. L. Meza, S. Coutermarsh-Ott, A. Hazy, D. Gris, and I. C. Allen, Loss of NLRX1 Exacerbates Neural Tissue Damage and NF-kappaB Signaling following Brain Injury. J Immunol, 2017. 199(10): p. 3547-3558.
Qin, Y., B. Xue, C. Liu, X. Wang, R. Tian, Q. Xie, M. Guo, G. Li, D. Yang, and H. Zhu, NLRX1 mediates MAVS degradation to attenuate hepatitis C virus-induced innate immune response through PCBP2. J Virol, 2017.
Wang, Y. G., W. L. Fang, J. Wei, T. Wang, N. Wang, J. L. Ma, and M. Shi, The involvement of NLRX1 and NLRP3 in the development of nonalcoholic steatohepatitis in mice. J Chin Med Assoc, 2013. 76(12): p. 686-692.

### EXEMPLARY EMBODIMENTS

1. A compound of Formula I:

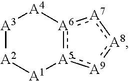

or a pharmaceutically acceptable salt or ester thereof, wherein:
A¹ and A⁴ are each independently C(R¹)₂, N(R¹), O, S, N(R^{O}), C(R¹)(R^{O}), or C(═O);
A² is C(R¹)₂, N(R¹), O, or S;
A³ is N(Y), C(R¹)(Y), N(L^{Q}-Y), or C(R¹)(L^{Q}-Y);
A⁵ and A⁶ are each independently C, C(R¹), or N;
A⁷, A⁸, and A⁹ are each independently C(R¹)₂, N(R¹), O, S, C(R¹), N, N(R^{A}), C(R¹)(R^{A}), C(R^{A}),
N(Z), C(R¹)(Z), C(Z), N(L^{Z}-Z), C(R¹)(L^{Z}-Z), or C(L^{Z}-Z), with the proviso that exactly one of A⁷, A⁸, and A⁹ is N(Z), C(R¹)(Z), C(Z), N(L^{Z}-Z), C(R¹)(L^{Z}-Z), or C(L^{Z}-Z);
R^{O} in each instance is independently hydroxyl, optionally substituted alkyloxy, thiol, optionally substituted alkylthio, or optionally substituted amino;
R^{A} is optionally substituted alkyl or hydroxyl;
L^{Q} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹), with the optional proviso that LQ is not an oxygen atom, a sulfur atom, or N(R¹) when A³ is N(L^{Q}-Y);
L^{Z} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹), with the optional proviso that L^{Z} is not an oxygen atom, a sulfur atom, or N(R¹) when the exactly one of A⁷, A⁸, or A⁹ is N(L^{Z}-Z);
Y is Y¹ or Y²;
Y¹ is:

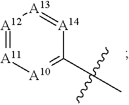

A¹⁰, A¹¹, A¹², A¹³, and A¹⁴ are each independently C(R¹), C(R^{Y}), or N, with the proviso that exactly one of A¹⁰, A¹¹, A¹², A¹³, and A¹⁴ is C(R^{Y});
Y² is:

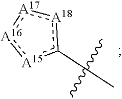

A¹⁵, A¹⁶, A¹⁷, and A¹⁸ are each independently C(R¹), C(R¹)₂, C(R^{Y}), C(R¹)(R^{Y}), N, N(R¹), N(R^{Y}), S, or O, with the proviso that exactly one of A¹⁵, A¹⁶, A¹⁷, and A¹⁸ is C(R^{Y}), C(R¹)(R^{Y}), or N(R^{Y});
R^{Y} is R^{L} or L^{Y}-R^{L};
R^{L} is hydroxyl, carboxyl, optionally substituted alkyloxy, thiol, sulfino, optionally substituted alkylthio, optionally substituted amino, optionally substituted alkyloxycarbonyl, optionally substituted carbamoyl, or optionally substituted sulfamoyl;
L^{Y} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹), with the optional proviso that L^{Y} is not an oxygen atom, a sulfur atom, or N(R¹) when the exactly one of A¹⁵, A¹⁶, A¹⁷, and A¹⁸ is N(R^{Y});
Z is:

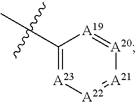

A¹⁹ and A²³ are each independently C(R¹) or N;
A²⁰, A²¹, and A²² are each independently C(R¹), N, or C(R^{Z});
R^{Z} in each instance is independently halogen, optionally substituted alkyl, hydroxyl, optionally substituted alkyloxy, thiol, or optionally substituted alkylthio;
each - - - between adjacent atoms represents a bond that is present or absent;
R¹ in each instance is independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclic group.
2. The compound of any prior embodiment, wherein at least one of A¹ and A⁴ is C(R¹)(R^{O}), or C(═O).
3. The compound of any prior embodiment, wherein A¹ is C(R¹)(R^{O}) or C(═O).
4. The compound of any prior embodiment, wherein A¹ is C(═O).
5. The compound of any one of embodiments 1-3, wherein A¹ is C(R¹)(R^{O}).
6. The compound of embodiment 5, wherein the R^{O} of A¹ is hydroxyl.
7. The compound of any one of embodiments 1-2, wherein A¹ is C(R¹)₂.
8. The compound of any prior embodiment, wherein A⁴ is C(R¹)(R^{O}), or C(═O).
9. The compound of any prior embodiment, wherein A⁴ is C(═O).
10. The compound of any one of embodiments 1-8, wherein A⁴ is C(R¹)(R^{O}).
11. The compound of embodiment 10, wherein the R^{O} of A⁴ is hydroxyl.
12. The compound of any one of embodiments 1-7, wherein A⁴ is C(R¹)₂.
13. The compound of any prior embodiment, wherein A² is C(R¹)₂.
14. The compound of any prior embodiment, wherein A³ is N(Y) or N(L^{Q}-Y).
15. The compound of any one of embodiments 1-13, wherein A³ is N(Y) or C(R¹)(Y).
16. The compound of any prior embodiment, wherein A³ is N(Y).
17. The compound of any prior embodiment, wherein A⁵ is C.
18. The compound of any prior embodiment, wherein A⁶ is C.
19. The compound of any one of embodiments 1-16, wherein A⁵ and A⁶ are each C.
20. The compound of any prior embodiment, wherein A⁷ is C(R¹) or C(R^{A}).
21. The compound of embodiment 20, wherein A⁷ is C(R¹).
22. The compound of embodiment 20, wherein A⁷ is C(R^{A}).
23. The compound of any prior embodiment, wherein R^{A} in each instance is unsubstituted C1-C4 alkyl, halogen-substituted C1-C4 alkyl, or hydroxyl.
24. The compound of any prior embodiment, wherein R^{A} in each instance is unsubstituted C1-C4 alkyl.
25. The compound of any prior embodiment, wherein A⁸ and A⁹ are each independently N(R¹), N, N(Z), or N(L^{Z}-Z).
26. The compound of any prior embodiment, wherein A⁸ and A⁹ are each independently N, N(Z), or N(L^{Z}-Z), with the proviso that exactly one of A⁸ and A⁹ is N(Z) or N(L^{Z}-Z).
27. The compound of any prior embodiment, wherein A⁸ is N(Z) or N(L^{Z}-Z).
28. The compound of any one of embodiments 1-26, wherein A⁹ is N(Z) or N(L^{Z}-Z).
29. The compound of any one of embodiments 1-25, wherein A⁸ and A⁹ are each independently N or N(Z), with the proviso that exactly one of A⁸ and A⁹ is N(Z).
30. The compound of embodiment 29, wherein A⁸ is N(Z).
31. The compound of embodiment 29, wherein A⁹ is N(Z).
32. The compound of any prior embodiment, wherein at least one of A¹⁰, A¹¹, A¹², A¹³, and A¹⁴ is N.
33. The compound of any prior embodiment, wherein exactly one of A¹⁰, A¹¹, A¹², A¹³, and A¹⁴ is N.
34. The compound of any prior embodiment, wherein A¹⁰ is N.
35. The compound of any one of embodiments 1-33, wherein A¹¹ is N.
36. The compound of any one of embodiments 1-33, wherein A¹² is N.
37. The compound of any one of embodiments 1-33, wherein A¹³ is N.
38. The compound of any one of embodiments 1-33, wherein A¹⁴ is N.
39. The compound of any one of embodiments 1-32, wherein A¹⁰ and A¹³ are N.
40. The compound of any one of embodiments 1-32, wherein A¹² and A¹⁴ are N.
41. The compound of any one of embodiments 1-33, wherein exactly one of A¹¹ and A¹² is C(R^{Y}).
42. The compound of embodiment 41, wherein A¹¹ is C(R^{Y}).
43. The compound of embodiment 41, wherein A¹² is C(R^{Y}).
44. The compound of any one of embodiments 41-43, wherein A¹³ is N.
45. The compound of any one of embodiments 41-44, wherein each of A¹⁰, A¹¹, A¹², A¹³, and A¹⁴ is C(R¹) unless specified otherwise.
46. The compound of any prior embodiment, wherein Y is Y¹.
47. The compound of any one of embodiments 1-45, wherein exactly one of A¹⁵, A¹⁶, A¹⁷, and A¹⁸ is N(R¹), N(R^{Y}), S, or O.
48. The compound of any prior embodiment, wherein at least one of A¹⁵, A¹⁶, A¹⁷, and A¹⁸ is N, N(R¹), N(R^{Y}), S, or O.
49. The compound of any prior embodiment, wherein at least two of A¹⁵, A¹⁶, A¹⁷, and A¹⁸ are each independently N, N(R¹), N(R^{Y}), S, or O.
50. The compound of any prior embodiment, wherein exactly one of A¹⁵, A¹⁶, and A¹⁷ is C(R^{Y}) or N(R^{Y}).
51. The compound of any prior embodiment, wherein A¹⁵ is N.
52. The compound of any one of embodiments 1-50, wherein A¹⁵ is C(R¹).
53. The compound of any prior embodiment, wherein A¹⁶ is C(R^{Y}) or N(R^{Y}).
54. The compound of any prior embodiment, wherein A¹⁷ is N(R¹), S, or O.
55. The compound of any prior embodiment, wherein A¹⁷ is N(R¹).
56. The compound of any prior embodiment, wherein A¹⁸ is N.
57. The compound of any one of embodiments 1-31 and 47-56, wherein Y is Y².
58. The compound of any prior embodiment, wherein R^{L} is hydroxyl, carboxyl, optionally substituted alkyloxy, optionally substituted amino, optionally substituted alkyloxycarbonyl, and optionally substituted carbamoyl.
59. The compound of any prior embodiment, wherein R^{L} is hydroxyl, carboxyl, unsubstituted C1-C4 alkyloxy, unsubstituted amino, amino substituted with one or two C1-C4 alkyl groups, unsubstituted C1-C4 alkyloxycarbonyl, unsubstituted carbamoyl, and carbamoyl comprising an amino substituted with one or two C1-C4 alkyl groups.
60. The compound of any prior embodiment, wherein R^{L} is carboxyl.
61. The compound of any prior embodiment, wherein R^{Y} is L^{Y}-R^{L}.
62. The compound of any prior embodiment, wherein L^{Y} is optionally substituted alkylene optionally containing one or two heteroatom(s).
63. The compound of any prior embodiment, wherein L^{Y} is optionally substituted alkylene optionally containing one or two heteroatom(s) with a contiguous backbone chain of no more than four atoms.
64. The compound of any prior embodiment, wherein L^{Y} is optionally substituted alkylene with a contiguous backbone chain of no more than four atoms.
65. The compound of any prior embodiment, wherein L^{Y} is unsubstituted C1, C2, C3, or C4 alkylene.
66. The compound of any one of embodiments 1-60, wherein R^{Y} is R^{L}.
67. The compound of any prior embodiment, wherein at least one of A¹⁹, A²⁰, A²¹, A²², and A²³ is N or at least one of A²⁰, A²¹, and A²² is C(R^{Z}).
68. The compound of any prior embodiment, wherein at least one of A¹⁹, A²⁰, A²¹, A²², and A²³ is N.
69. The compound of prior embodiment, wherein A¹⁹ is N.
70. The compound of any one of embodiments 1-68, wherein A²⁰ is N.
71. The compound of any one of embodiments 1-68, wherein A²¹ is N.
72. The compound of any one of embodiments 1-68, wherein A²⁰ and A²² are each N.
73. The compound of any one of embodiments 1-67, wherein at least one of A²⁰, A²¹, and A²² is C(R^{Z}).
74. The compound of any one of embodiments 1-68 and 73, wherein at least two of A²⁰, A²¹, and A²² are C(R^{Z}).
75. The compound of any one of embodiments 1-68 and 73-74, wherein A²⁰ is C(R^{Z}).
76. The compound of any one of embodiments 1-68 and 73-75, wherein A²¹ is C(R^{Z}).
77. The compound of any one of embodiments 1-68 and 73-76, wherein A²² is C(R^{Z}).
78. The compound of any one of embodiments 1-68 and 73-77, wherein A¹⁹ and A²³ are each C(R¹).
79. The compound of any prior embodiment, wherein R^{Z} in each instance is independently halogen, optionally substituted alkyl, hydroxyl, or optionally substituted alkyloxy.
80. The compound of any prior embodiment, wherein R^{Z} in each instance is independently halogen, unsubstituted C1-C4 alkyl, halogen-substituted C1-C4 alkyl, hydroxyl, or unsubstituted C1-C4 alkyloxy.
81. The compound of any prior embodiment, wherein R^{Z} in each instance is halogen.
82. The compound of any prior embodiment, wherein R¹ in each instance, except where specified otherwise, is independently hydrogen, halogen, optionally substituted alkyl, optionally substituted cycloalkyl, optionally substituted alkyloxy, optionally substituted cycloalkyloxy, optionally substituted alkylthio, optionally substituted alkylsulfinyl, optionally substituted cycloalkylthio, optionally substituted cycloalkylsulfinyl, optionally substituted amino, acyl, optionally substituted aryl, optionally substituted aryloxy, optionally substituted arylthio, optionally substituted heteroaryl, optionally substituted heteroaryloxy, optionally substituted heteroarylthio, optionally substituted heteroarylsulfinyl, or an optionally substituted non-aromatic heterocyclic group.
83. The compound of any prior embodiment, wherein R¹ in each instance, except where specified otherwise, is independently hydrogen, halogen, unsubstituted alkyl, unsubstituted cycloalkyl, unsubstituted alkyloxy, unsubstituted cycloalkyloxy, unsubstituted alkylthio, unsubstituted alkylsulfinyl, unsubstituted cycloalkylthio, unsubstituted cycloalkylsulfinyl, unsubstituted amino, acyl, unsubstituted aryl, unsubstituted aryloxy, unsubstituted arylthio, unsubstituted heteroaryl, unsubstituted heteroaryloxy, unsubstituted heteroarylthio, unsubstituted heteroarylsulfinyl, or an unsubstituted non-aromatic heterocyclic group.
84. The compound of any prior embodiment, wherein R¹ in each instance, except where specified otherwise, is independently hydrogen or halogen.
85. The compound of any prior embodiment, wherein RI in each instance, except where specified otherwise, is hydrogen.
86. The compound of embodiment 1, wherein the compound has the structure of any compound shown in FIGS. 1A-1R, or a pharmaceutically acceptable salt or ester thereof.
87. The compound of embodiment 1, wherein the compound has the structure of any compound shown in FIGS. 2A-2E, or a pharmaceutically acceptable salt or ester thereof.
88. The compound of embodiment 1, wherein the compound has the structure of:

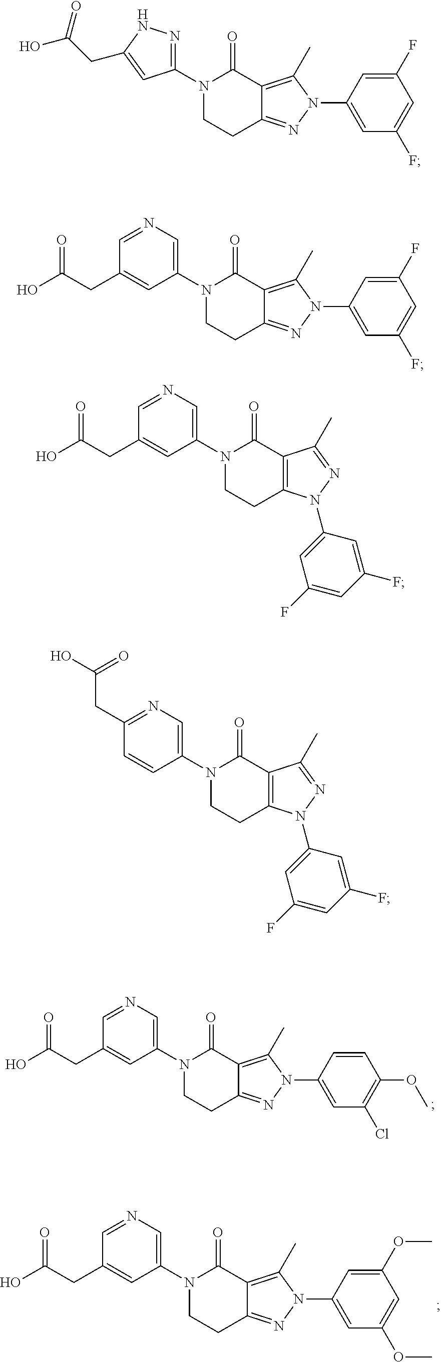 [SMILES: Cc1c2c(nn1-c1cc(F)cc(F)c1)CCN(c1cc(CC(=O)O)[nH]n1)C2=O ; Cc1c2c(nn1-c1cc(F)cc(F)c1)CCN(c1cncc(CC(=O)O)c1)C2=O ; Cc1nn(-c2cc(F)cc(F)c2)c2c1C(=O)N(c1cncc(CC(=O)O)c1)CC2 ; Cc1nn(-c2cc(F)cc(F)c2)c2c1C(=O)N(c1ccc(CC(=O)O)nc1)CC2 ; COc1ccc(-n2nc3c(c2C)C(=O)N(c2cncc(CC(=O)O)c2)CC3)cc1Cl ; COc1cc(OC)cc(-n2nc3c(c2C)C(=O)N(c2cncc(CC(=O)O)c2)CC3)c1]

or a pharmaceutically acceptable salt or ester thereof.
89. A method of treating a condition in an animal with a compound as recited in any prior embodiment, the method comprising administering an effective amount of the compound to the animal, wherein the condition comprises at least one of a chronic and/or inflammatory respiratory disease, a chronic and/or inflammatory disease of the central nervous system, an allergic disease, an autoimmune disease, a cardiovascular disease, diabetes, hypereosinophilic syndrome, a granulomatous disorder, cancer, and an infectious disease.
90. The method of embodiment 89, wherein the condition comprises a chronic and/or inflammatory respiratory disease.
91. The method of embodiment 90, wherein the chronic and/or inflammatory respiratory disease comprises asthma.
92. The method of embodiment 91, wherein the asthma comprises chronic asthma.
93. The method of embodiment 91, wherein the asthma comprises acute asthma.
94. The method of embodiment 91, wherein the asthma comprises allergic asthma.
95. The method of embodiment 91, wherein the asthma comprises type 2 or eosinophilic asthma.
96. The method of embodiment 91, wherein the asthma comprises non-type 2 or neutrophilic asthma.
97. The method of embodiment 90, wherein the chronic and/or inflammatory respiratory disease comprises chronic obstructive pulmonary disease.
98. The method of embodiment 90, wherein the chronic and/or inflammatory respiratory disease comprises pulmonary fibrosis.
99. The method of embodiment 89, wherein condition comprises a chronic and/or inflammatory disease of the central nervous system
100. The method of embodiment 99, wherein the chronic and/or inflammatory disease of the central nervous system comprises Alzheimer's disease.
101. The method of embodiment 89 wherein condition comprises an allergic disease.
102. The method of embodiment 101, wherein the allergic disease comprises an allergic disorder of the skin.
103. The method of embodiment 102, wherein the allergic disorder of the skin comprises atopic dermatitis.
104. The method of embodiment 101, wherein the allergic disease comprises an eosinophilic gastrointestinal disorder.
105. The method of embodiment 104, wherein the eosinophilic gastrointestinal disorder comprises eosinophilic gastroenteritis or eosinophilic esophagitis.
106. The method of embodiment 89, wherein the condition comprises diabetes.
107. The method of embodiment 106, wherein the diabetes comprises type 2 diabetes.
108. The method of embodiment 89, wherein condition comprises a granulomatous disorder.
109. The method of embodiment 108, wherein the granulomatous disorder comprises Churg-Strauss syndrome.
110. The method of embodiment 108, wherein the granulomatous disorder comprises berylliosis.
111. The method of embodiment 108, wherein the granulomatous disorder comprises sarcoidosis.

1. A compound of Formula I: 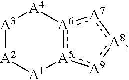
or a pharmaceutically acceptable salt or ester thereof, wherein:
A¹ and A⁴ are each independently C(R¹)₂, N(R¹), O, S, N(R^{O}), C(R¹)(R^{O}), or C(═O);
A² is C(R¹)₂, N(R¹), O, or S;
A³ is N(Y), C(R¹)(Y), N(L^{Q}-Y), or C(R¹)(L^{Q}-Y);
A⁵ and A⁶ are each independently C, C(R¹), or N;
A⁷, A⁸, and A⁹ are each independently C(R¹)₂, N(R¹), O, S, C(R¹), N, N(R^{A}), C(R¹)(R^{A}), C(R^{A}), N(Z), C(R¹)(Z), C(Z), N(L^{Z}-Z), C(R¹)(L^{Z}-Z), or C(L^{Z}-Z), with the proviso that exactly one of A⁷, A⁸, and A⁹ is N(Z), C(R¹)(Z), C(Z), N(L^{Z}-Z), C(R¹)(L^{Z}-Z), or C(L^{Z}-Z);
R^{O} in each instance is independently hydroxyl, optionally substituted alkyloxy, thiol, optionally substituted alkylthio, or optionally substituted amino;
R^{A} is optionally substituted alkyl or hydroxyl;
L^{Q} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹);
L^{Z} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹);
Y is Y¹ or Y²;
Y¹ is: 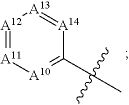
A¹⁰, A¹¹, A¹², A¹³, and A¹⁴ are each independently C(R¹), C(R^{Y}), or N, with the proviso that exactly one of A¹⁰, A¹¹, A¹², A¹³, and A¹⁴ is C(R^{Y});
Y² is: 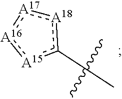
A¹⁵, A¹⁶, A¹⁷, and A¹⁸ are each independently C(R¹), C(R¹)₂, C(R^{Y}), C(R¹)(R^{Y}), N, N(R¹), N(R^{Y}), S, or O, with the proviso that exactly one of A¹⁵, A¹⁶, A¹⁷, and A¹⁸ is C(R^{Y}), C(R¹)(R^{Y}), or N(R^{Y});
R^{Y} is R^{L} or L^{Y}-R^{L};
R^{L} is hydroxyl, carboxyl, optionally substituted alkyloxy, thiol, sulfino, optionally substituted alkylthio, optionally substituted amino, optionally substituted alkyloxycarbonyl, optionally substituted carbamoyl, or optionally substituted sulfamoyl;
L^{Y} is optionally substituted alkylene optionally containing one or two heteroatom(s), optionally substituted alkenylene optionally containing one or two heteroatom(s), optionally substituted alkynylene optionally containing one or two heteroatom(s), an oxygen atom, a sulfur atom, or N(R¹);
Z is: 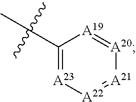
A¹⁹ and A²³ are each independently C(R¹) or N;
A²⁰, A²¹, and A²² are each independently C(R¹), N, or C(R^{Z});
R^{Z} in each instance is independently halogen, optionally substituted alkyl, hydroxyl, optionally substituted alkyloxy, thiol, or optionally substituted alkylthio;
each - - - between adjacent atoms represents a bond that is present or absent;
R¹ in each instance is independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclic group.

2. The compound of claim 1, wherein at least one of A¹ and A⁴ is C(R¹)(R^{O}), or C(═O).

3. (canceled)

4. The compound of claim 1, wherein A³ is N(Y) or N(L^{Q}-Y).

5. The compound of claim 1, wherein A⁸ and A⁹ are each independently N(R¹), N, N(Z), or N(L^{Z}-Z).

6. (canceled)

7. The compound of claim 1, wherein A⁵ and A⁶ are each C.

8. The compound of claim 1, wherein:
A¹ and A² are each C(R¹)₂; and
A⁷ is C(R¹) or C(R^{A}).

9. (canceled)

10. The compound of claim 1, wherein A¹³ is N.

11. The compound of claim 9any prior claim, wherein:
A¹⁰ and A¹⁴ are each C(R¹); and
the one of A¹¹ and A¹² that is not C(R^{Y}) is C(R¹).

12. The compound of claim 1, wherein Y is Y^{1.}

13. (canceled) 14 (Currently Amended) The compound of claim 1, wherein at least two of A¹⁵, A¹⁶, A¹⁷, and A¹⁸ are each independently N, N(R¹), N(R^{Y}), S, or O.

15. (canceled)

16. The compound of claim 1, wherein A¹⁶ is C(R^{Y}).

17. The compound of claim 1, wherein A¹⁸ is N.

18. The compound of claim 1, wherein Y is Y².

19. The compound of claim 1, wherein R^{L} is hydroxyl, carboxyl, optionally substituted alkyloxy, optionally substituted amino, optionally substituted alkyloxycarbonyl, and optionally substituted carbamoyl.

20. (canceled)

21. The compound of claim 1, wherein R^{Y} is L^{Y}-R_{L}.

22. to 23. (canceled)

24. The compound of claim 1, wherein R^{Z} in each instance is independently halogen, optionally substituted alkyl, hydroxyl, or optionally substituted alkyloxy.

25. The compound of claim 1, wherein R¹ in each instance is independently hydrogen, halogen, unsubstituted alkyl, unsubstituted cycloalkyl, unsubstituted alkyloxy, unsubstituted cycloalkyloxy, unsubstituted alkylthio, unsubstituted alkylsulfinyl, unsubstituted cycloalkylthio, unsubstituted cycloalkylsulfinyl, unsubstituted amino, acyl, unsubstituted aryl, unsubstituted aryloxy, unsubstituted arylthio, unsubstituted heteroaryl, unsubstituted heteroaryloxy, unsubstituted heteroarylthio, unsubstituted heteroarylsulfinyl, or an unsubstituted non-aromatic heterocyclic group.

26. to 27. (canceled)

28. The compound of claim 1, wherein the compound has the structure of: 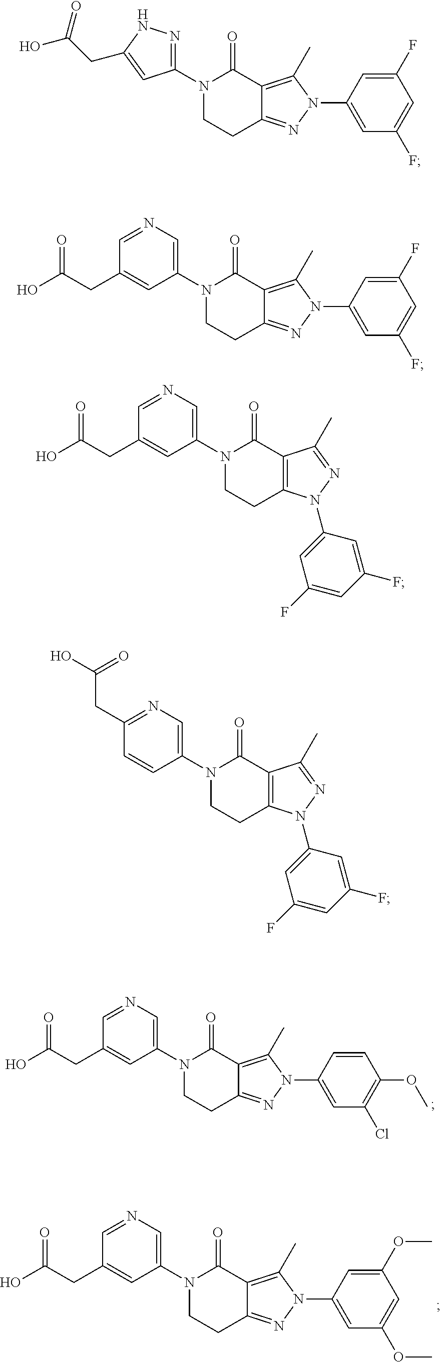 [SMILES: Cc1c2c(nn1-c1cc(F)cc(F)c1)CCN(c1cc(CC(=O)O)[nH]n1)C2=O ; Cc1c2c(nn1-c1cc(F)cc(F)c1)CCN(c1cncc(CC(=O)O)c1)C2=O ; Cc1nn(-c2cc(F)cc(F)c2)c2c1C(=O)N(c1cncc(CC(=O)O)c1)CC2 ; Cc1nn(-c2cc(F)cc(F)c2)c2c1C(=O)N(c1ccc(CC(=O)O)nc1)CC2 ; COc1ccc(-n2nc3c(c2C)C(=O)N(c2cncc(CC(=O)O)c2)CC3)cc1Cl ; COc1cc(OC)cc(-n2nc3c(c2C)C(=O)N(c2cncc(CC(=O)O)c2)CC3)c1]
or a pharmaceutically acceptable salt or ester thereof.

29. A method of treating a condition in an animal with a compound as recited in claim 1, the method comprising administering an effective amount of the compound to the animal, wherein the condition comprises a chronic and/or inflammatory respiratory disease, a chronic and/or inflammatory disease of the central nervous system, an allergic disease, an autoimmune disease, a cardiovascular disease, diabetes, hypereosinophilic syndrome, a granulomatous disorder, cancer, or an infectious disease, or a combination of any of the foregoing.

30. (canceled)

31. The method of claim 29, wherein the condition comprises one or more of asthma, chronic obstructive pulmonary disease, pulmonary fibrosis, Alzheimer's disease, atopic dermatitis, eosinophilic gastroenteritis, eosinophilic esophagitis, diabetes, or a granulomatous disorder comprising Churg-Strauss syndrome, berylliosis, or sarcoidosis, or a combination thereof.
